(19) United States
(12) Patent Application Publication
(10) Pub. No.: US 2016/0115164 A1
(43) Pub. Date: Apr. 28, 2016

(54) BICYCLIC HETEROCYCLES AS FGFR4 INHIBITORS

(71) Applicant: Incyte Corporation, Wilmington, DE (US)

(72) Inventors: Liangxing Wu, Wilmington, DE (US); Liang Lu, Hockessin, DE (US); Ding-Quan Qian, Newark, DE (US); Bo Shen, Garnet Valley, PA (US); Wenqing Yao, Chadds Ford, PA (US)

(21) Appl. No.: 14/918,868

(22) Filed: Oct. 21, 2015

Related U.S. Application Data
(60) Provisional application No. 62/118,712, filed on Feb. 20, 2015.
(60) Provisional application No. 62/067,237, filed on Oct. 22, 2014.

(51) Int. Cl. C07D 471/10; C07D 491/20

(52) U.S. Cl. CPC C07D 471/10; C07D 491/20

(57) ABSTRACT

The present invention relates to bicyclic heterocycles, and pharmaceutical compositions of the same, that are inhibitors of the FGFR4 enzyme and are useful in the treatment of FGFR4-associated diseases such as cancer.

### FIELD OF THE INVENTION

The present invention relates to bicyclic heterocycles, and pharmaceutical compositions of the same, that are inhibitors of the enzyme FGFR4 and are useful in the treatment of FGFR4-associated diseases such as cancer.

### BACKGROUND OF INVENTION

The Fibroblast Growth Factor Receptors (FGFR) are receptor tyrosine kinases that bind to fibroblast growth factor (FGF) ligands. There are four FGFR proteins (FGFR1-4) that are capable of binding ligands and are involved in the regulation of many physiological processes including tissue development, angiogenesis, wound healing, and metabolic regulation. Upon ligand binding, the receptors undergo dimerization and phosphorylation leading to stimulation of the protein kinase activity and recruitment of many intracellular docking proteins. These interactions facilitate the activation of an array of intracellular signaling pathways including Ras-MAPK, AKT-PI3K, and phospholipase C that are important for cellular growth, proliferation and survival (Reviewed in Eswarakumar et al. Cytokine & Growth Factor Reviews, 2005).

Aberrant activation of this pathway either through overexpression of FGF ligands or FGFR or activating mutations in the FGFRs can lead to tumor development, progression, and resistance to conventional cancer therapies. In human cancer, genetic alterations including gene amplification, chromosomal translocations and somatic mutations that lead to ligand-independent receptor activation have been described. Large scale DNA sequencing of thousands of tumor samples has revealed that components of the FGFR pathway are among the most frequently mutated in human cancer. Many of these activating mutations are identical to germline mutations that lead to skeletal dysplasia syndromes. Mechanisms that lead to aberrant ligand-dependent signaling in human disease include overexpression of FGFs and changes in FGFR splicing that lead to receptors with more promiscuous ligand binding abilities (Reviewed in Knights and Cook Pharmacology & Therapeutics, 2010; Turner and Grose, Nature Reviews Cancer, 2010). Therefore, development of inhibitors targeting FGFR may be useful in the clinical treatment of diseases that have elevated FGF or FGFR activity.

The cancer types in which FGF/FGFRs are implicated include, but are not limited to: carcinomas (e.g., bladder, breast, cervical, colorectal, endometrial, gastric, head and neck, kidney, liver, lung, ovarian, prostate); hematopoietic malignancies (e.g., multiple myeloma, chronic lymphocytic lymphoma, adult T cell leukemia, acute myelogenous leukemia, non-Hodgkin lymphoma, myeloproliferative neoplasms, and Waldenstrom's Macroglubulinemia); and other neoplasms (e.g., glioblastoma, melanoma, and rhabdosarcoma). In addition to a role in oncogenic neoplasms, FGFR activation has also been implicated in skeletal and chondrocyte disorders including, but not limited to, achrondroplasia and craniosynostosis syndromes.

The FGFR4-FGF19 signaling axis, specifically, has been implicated in the pathogenesis of a number of cancers including hepatocellular carcinoma (Heinzle et al., Cur. Pharm. Des. 2014, 20:2881). Ectopic expression of FGF19 in transgenic mice was shown to lead to tumor formation in the liver and a neutralizing antibody to FGF19 was found to inhibit tumor growth in mice. In addition, overexpression of FGFR4 has been observed in a multiple tumor types including hepatocellular carcinoma, colorectal, breast, pancreatic, prostate, lung, and thyroid cancers. Furthermore, activating mutations in FGFR4 have been reported in rhabdomyosarcoma (Taylor et al. JCI 2009, 119:3395). Targeting FGFR4 with selective small molecule inhibitors may therefore prove beneficial in the treatment of certain cancers.

There is a continuing need for the development of new drugs for the treatment of cancer and other diseases, and the FGFR4 inhibitors described herein help address this need.

### SUMMARY OF INVENTION

The present invention is directed to inhibitors of FGFR4 having Formula (I′):

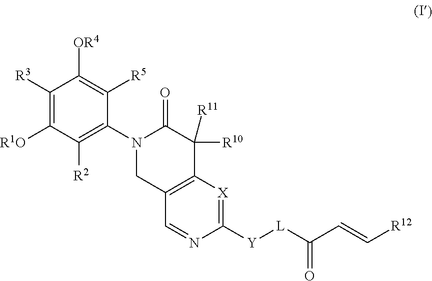

or a pharmaceutically acceptable salt thereof, wherein constituent variables are defined herein. The present invention is further directed to pharmaceutical compositions comprising a compound of Formula (I′), or a pharmaceutically acceptable salt thereof, and at least one pharmaceutically acceptable carrier.

The present invention is further directed to methods of inhibiting an FGFR4 enzyme comprising contacting the enzyme with a compound of Formula (I′), or a pharmaceutically acceptable salt thereof.

The present invention is further directed to a method of treating a disease associated with abnormal activity or expression of an FGFR4 enzyme, comprising administering a compound of Formula (I′), or a pharmaceutically acceptable salt thereof, to a patient in need thereof.

The present invention is further directed to compounds of Formula (I′) for use in treating a disease associated with abnormal activity or expression of an FGFR4 enzyme.

The present invention is further directed to a method for treating a disorder mediated by an FGFR4 enzyme, or a mutant thereof, in a patient in need thereof, comprising the step of administering to said patient a compound according to the present invention or pharmaceutically acceptable composition thereof.

The present invention is further directed to the use of compounds of Formula (I′) in the preparation of a medicament for use in therapy.

### DETAILED DESCRIPTION

In one aspect, the present invention provides compounds of Formula (I′):

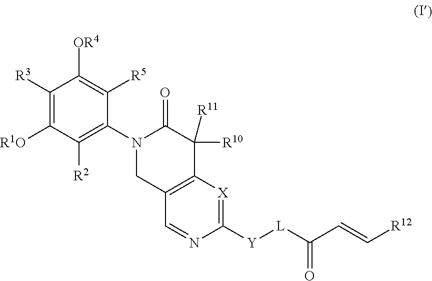

or a pharmaceutically acceptable salt thereof, wherein:

X is N or CR⁶;

Y is O or NR⁸;

R¹ is C₁₋₃ alkyl or C₁₋₃ haloalkyl;

R² is H, halo, C₁₋₃ alkyl, C₁₋₃ haloalkyl, CN, or C₁₋₃ alkoxy;

R³ is H, halo, C₁₋₃ alkyl, C₁₋₃ haloalkyl, CN, or C₁₋₃ alkoxy;

R⁴ is C₁₋₃ alkyl or C₁₋₃ haloalkyl;

R⁵ is H, halo, C₁₋₃ alkyl, C₁₋₃ haloalkyl, CN, or C₁₋₃ alkoxy;

R⁶ is H, halo, CN, OR^{a4}, SR^{a4}, C(O)NR^{c4}R^{d4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, C₁₋₆ alkyl, C₂₋₆ alkenyl, C₂₋₆ alkynyl, C₁₋₆ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, phenyl, C₃₋₆ cycloalkyl, 5-6 membered heteroaryl, and 4-7 membered heterocycloalkyl groups of R⁶ are each optionally substituted with 1, 2, or 3 substituents independently selected from R^{10A};

L is a bond or *—CR^{7A}R^{7B} [symbol] CR^{7C}R^{7D}—NR^{7E}—, wherein the symbol * indicates the point of attachment to Y in Formula (I′); wherein the symbol [symbol] represents a single or double bond; and wherein R^{7B} and R^{7D} are absent when [symbol] is a double bond;

R^{7A} is selected from H, C₁₋₄ alkyl, C₂₋₄ alkenyl, C₂₋₄ alkynyl, phenyl, C₃₋₇ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₇ cycloalkyl, 5-6 membered heteroaryl, and 4-7 membered heterocycloalkyl groups of R^{7A} are each optionally substituted with 1, 2, or 3 substituents independently selected from R¹⁷;

R^{7B} is H or C₁₋₄ alkyl optionally substituted with 1, 2, or 3 substituents independently selected from R¹⁷;

or R^{7A} and R^{7B} together with the carbon atom to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl and 4-7 membered heterocycloalkyl groups are each optionally substituted with 1, 2, or 3 substituents independently selected from R¹⁷;

R^{7C} is selected from H, C₁₋₄ alkyl, C₂₋₄ alkenyl, C₂₋₄ alkynyl, phenyl, C₃₋₇ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₇ cycloalkyl, 5-6 membered heteroaryl, and 4-7 membered heterocycloalkyl group of R^{7C} are each optionally substituted with 1, 2, or 3 substituents independently selected from R¹⁷;

R^{7D} is H or C₁₋₄ alkyl optionally substituted with 1, 2, or 3 substituents independently selected from R¹⁷;

or R^{7C} and R^{7D} together with the carbon atom to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl and 4-7 membered heterocycloalkyl groups are each optionally substituted with 1, 2, or 3 substituents independently selected from R¹⁷;

R^{7E} is selected from H, C₁₋₄ alkyl, C₂₋₄ alkenyl, and C₂₋₄ alkynyl;

alternatively, when [symbol] is a double bond, R^{7A} and R^{7C} together with the carbon atoms to which they are attached form a phenyl group or a 5-6 membered heteroaryl moiety comprising carbon and 1, 2, or 3 heteroatoms independently selected from N, O and S, wherein said phenyl and 5-6 membered heteroaryl groups are each optionally substituted with 1, 2, or 3 substituents independently selected from R¹⁷;

alternatively, when [symbol] is a single bond, R^{7A} and R^{7C} together with the carbon atoms to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl group and 4-7 membered heterocycloalkyl group are each optionally substituted with 1, 2, or 3 substituents independently selected from R¹⁷;

alternatively, R^{7A} and R^{7E} together with the carbon and nitrogen atoms to which they are attached form a 5-6 membered heteroaryl moiety comprising carbon and 1, 2, or 3 nitrogen atoms, or a 4-7 membered heterocycloalkyl moiety comprising carbon and 1 or 2 nitrogen atoms; wherein said 5-6 membered heteroaryl and 4-7 membered heterocycloalkyl group are each optionally substituted with 1, 2, or 3 substituents independently selected from R¹⁷;

alternatively, R^{7C} and R^{7E} together with the carbon and nitrogen atoms to which they are attached form a 5-6 membered heteroaryl moiety comprising carbon and 1, 2, or 3 nitrogen atoms, or a 4-7 membered heterocycloalkyl moiety comprising carbon and 1 or 2 nitrogen atoms; wherein said 5-6 membered heteroaryl and 4-7 membered heterocycloalkyl group are each optionally substituted with 1, 2, or 3 substituents independently selected from R¹⁷;

R⁸ is H or C₁₋₄ alkyl which is optionally substituted by halo, CN, OR^{a9}, C(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, S(O)₂NR^{c9}R^{d9}, phenyl, C₃₋₇ cycloalkyl, a 5-6 membered heteroaryl moiety comprising carbon and 1, 2, or 3 heteroatoms independently selected from N, O and S, or a 4-7 membered heterocycloalkyl moiety comprising carbon and 1, 2, or 3 heteroatoms independently selected from N, O and S; wherein said phenyl, C₃₋₇ cycloalkyl, 5-6 membered heteroaryl, and 4-7 membered heterocycloalkyl groups of R⁸ are each optionally substituted with 1 or 2 R¹⁹;

R¹⁰ is selected from C₁₋₆ alkyl, C₂₋₆ alkenyl, C₂₋₆ alkynyl, C₁₋₆ haloalkyl, C₆₋₁₀ aryl, C₃₋₁₀ cycloalkyl, a 5-10 membered heteroaryl moiety comprising carbon and 1, 2, or 3 heteroatoms independently selected from N, O and S, and a 4-10 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl groups of R¹⁰ are each optionally substituted with 1, 2, 3, or 4 R^{10A};

R^{10A}, at each occurrence, is independently selected from halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4} S(O)₂NR^{c4}R^{d4}, C₁₋₆ alkyl, C₂₋₆ alkenyl, C₂₋₆ alkynyl, C₁₋₆ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, phenyl, C₃₋₆ cycloalkyl, 5-6 membered heteroaryl, and 4-7 membered heterocycloalkyl group of R^{10A} are each optionally substituted with 1, 2, or 3 substituents independently selected from R¹⁹;

R^{a4}, R^{b4}, R^{c4}, and R^{d4}, at each occurrence, are independently selected from H, C₁₋₄ alkyl, C₂₋₄ alkenyl, C₂₋₄ alkynyl, C₁₋₄ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₆ cycloalkyl, 5-6 membered heteroaryl, and 4-7 membered heterocycloalkyl group of R^{a4}, R^{b4}, R^{c4}, and R^{d4} are each optionally substituted with 1, 2, or 3 substituents independently selected from R¹⁹;

alternatively, R^{c4} and R^{d4} together with the nitrogen atom to which they are attached form a 4-, 5-, 6-, or 7-membered heterocycloalkyl group which is optionally substituted with 1, 2 or 3 substituents independently selected from R¹⁹;

R^{e4} is H or C₁₋₄ alkyl;

R¹¹ is selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl, and C₂₋₆ alkynyl are each optionally substituted with 1, 2 or 3 substituents independently selected from R¹⁹;

alternatively, R¹⁰ and R¹¹ together with the carbon atom to which they are attached form a 3-, 4-, 5-, 6-, or 7-membered cycloalkyl group or a 4-, 5-, 6-, 7-, 8-, 9-, or 10-membered heterocycloalkyl group; wherein said 3-, 4-, 5-, 6-, or 7-membered cycloalkyl group and 4-, 5-, 6-, 7-, 8-, 9-, or 10-membered heterocycloalkyl group are each optionally substituted with 1, 2, 3 or 4 R^{10A};

R¹² is H or C₁₋₄ alkyl which is optionally substituted by R¹⁷;

R¹⁷, at each occurrence, is independently selected from halo, CN, NO₂, OR^{a7}, SR^{a7}, C(O)R^{b7}, C(O)NR^{c7}R^{d7}, C(O)OR^{a7}, OC(O)R^{b7}, OC(O)NR^{c7}R^{d7}, C(═NR^{e7})NR^{c7}R^{d7}, NR^{c7}C(═NR^{e7})NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)R^{b7}, NR^{c7}S(O)₂R^{b7}, NR^{c7}S(O)₂NR^{c7}R^{d7}, S(O)R^{b7}, S(O)NR^{c7}R^{d7}, S(O)₂R^{b7}, S(O)₂NR^{c7}R^{d7}, C₁₋₆ alkyl, C₂₋₆ alkenyl, C₂₋₆ alkynyl, C₁₋₆ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, phenyl, C₃₋₆ cycloalkyl, 5-6 membered heteroaryl, and 4-7 membered heterocycloalkyl groups of R¹⁷ are each optionally substituted with 1, 2, or 3 substituents independently selected from R¹⁹;

R^{a7}, R^{b7}, R^{c7}, and R^{d7}, at each occurrence, are independently selected from H, C₁₋₄ alkyl, C₂₋₄ alkenyl, C₂₋₄ alkynyl, C₁₋₄ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₆ cycloalkyl, 5-6 membered heteroaryl, and 4-7 membered heterocycloalkyl groups of R^{a7}, R^{b7}, R^{c7}, and R^{d7} are each optionally substituted with 1, 2, or 3 substituents independently selected from R¹⁹;

alternatively, R^{c7} and R^{d7} together with the nitrogen atom to which they are attached form a 4-, 5-, 6-, or 7-membered heterocycloalkyl group which is optionally substituted with 1, 2 or 3 substituents independently selected from R¹⁹;

R^{e7} is H or C₁₋₄ alkyl;

R¹⁹, at each occurrence, is independently selected from halo, CN, NO₂, OR^{a9}, SR^{a9}, C(O)R^{b9}, C(O)NR^{c9}R^{d9}, C(O)OR^{a9}, OC(O)R^{b9}, OC(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, S(O)₂NR^{c9}R^{d9}, C₁₋₄ alkyl, C₂₋₄ alkenyl, C₂₋₄ alkynyl, and C₁₋₄ haloalkyl;

R^{a9}, R^{c9}, and R^{d9}, at each occurrence, are independently selected from H and C₁₋₄ alkyl; and

R^{b9} is C₁₋₄ alkyl. In one embodiment, Y is O. In another embodiment, Y is NR⁸.

In some embodiments, the present invention provides an inhibitor of FGFR4 which is a compound having Formula (I):

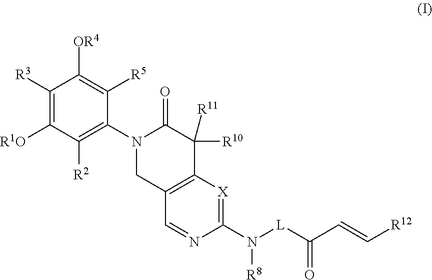

or a pharmaceutically acceptable salt thereof, wherein:

X is N or CR⁶;

R¹ is C₁₋₃ alkyl or C₁₋₃ haloalkyl;

R² is H, halo, C₁₋₃ alkyl, C₁₋₃ haloalkyl, CN, or C₁₋₃ alkoxy;

R³ is H, halo, C₁₋₃ alkyl, C₁₋₃ haloalkyl, CN, or C₁₋₃ alkoxy;

R⁴ is C₁₋₃ alkyl or C₁₋₃ haloalkyl;

R⁵ is H, halo, C₁₋₃ alkyl, C₁₋₃ haloalkyl, CN, or C₁₋₃ alkoxy;

R⁶ is H, halo, CN, OR^{a4}, SR^{a4}, C(O)NR^{c4}R^{d4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, C₁₋₆ alkyl, C₂₋₆ alkenyl, C₂₋₆ alkynyl, C₁₋₆ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, phenyl, C₃₋₆ cycloalkyl, 5-6 membered heteroaryl, and 4-7 membered heterocycloalkyl groups of R⁶ are each optionally substituted with 1, 2, or 3 substituents independently selected from R^{10A};

L is a bond or *—CR^{7A}R^{7B} [symbol] CR^{7C}R^{7D}—NR^{7E}—, wherein the symbol * indicates the point of attachment to NR⁸ in Formula (I); wherein the symbol [symbol] represents a single or double bond; and wherein R^{7B} and R^{7D} are absent when [symbol] is a double bond;

R^{7A} is selected from H, C₁₋₄ alkyl, C₂₋₄ alkenyl, C₂₋₄ alkynyl, phenyl, C₃₋₇ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₇ cycloalkyl, 5-6 membered heteroaryl, and 4-7 membered heterocycloalkyl groups of R^{7A} are each optionally substituted with 1, 2, or 3 substituents independently selected from R¹⁷;

R^{7B} is H or C₁₋₄ alkyl optionally substituted with 1, 2, or 3 substituents independently selected from R¹⁷;

or R^{7A} and R^{7B} together with the carbon atom to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl and 4-7 membered heterocycloalkyl groups are each optionally substituted with 1, 2, or 3 substituents independently selected from R¹⁷;

R^{7C} is selected from H, C₁₋₄ alkyl, C₂₋₄ alkenyl, C₂₋₄ alkynyl, phenyl, C₃₋₇ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₇ cycloalkyl, 5-6 membered heteroaryl, and 4-7 membered heterocycloalkyl group of R^{7C} are each optionally substituted with 1, 2, or 3 substituents independently selected from R¹⁷;

R^{7D} is H or C₁₋₄ alkyl optionally substituted with 1, 2, or 3 substituents independently selected from R¹⁷;

or R^{7C} and R^{7D} together with the carbon atom to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl and 4-7 membered heterocycloalkyl groups are each optionally substituted with 1, 2, or 3 substituents independently selected from R¹⁷;

R^{7E} is selected from H, C₁₋₄ alkyl, C₂₋₄ alkenyl, and C₂₋₄ alkynyl;

alternatively, when [symbol] is a double bond, R^{7A} and R^{7C} together with the carbon atoms to which they are attached form a phenyl group or a 5-6 membered heteroaryl moiety comprising carbon and 1, 2, or 3 heteroatoms independently selected from N, O and S, wherein said phenyl and 5-6 membered heteroaryl groups are each optionally substituted with 1, 2, or 3 substituents independently selected from R¹⁷;

alternatively, when [symbol] is a single bond, R^{7A} and R^{7C} together with the carbon atoms to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl group and 4-7 membered heterocycloalkyl group are each optionally substituted with 1, 2, or 3 substituents independently selected from R¹⁷;

alternatively, R^{7A} and R^{7E} together with the carbon and nitrogen atoms to which they are attached form a 5-6 membered heteroaryl moiety comprising carbon and 1, 2, or 3 nitrogen atoms, or a 4-7 membered heterocycloalkyl moiety comprising carbon and 1 or 2 nitrogen atoms; wherein said 5-6 membered heteroaryl and 4-7 membered heterocycloalkyl group are each optionally substituted with 1, 2, or 3 substituents independently selected from R¹⁷;

alternatively, R^{7C} and R^{7E} together with the carbon and nitrogen atoms to which they are attached form a 5-6 membered heteroaryl moiety comprising carbon and 1, 2, or 3 nitrogen atoms, or a 4-7 membered heterocycloalkyl moiety comprising carbon and 1 or 2 nitrogen atoms; wherein said 5-6 membered heteroaryl and 4-7 membered heterocycloalkyl group are each optionally substituted with 1, 2, or 3 substituents independently selected from R¹⁷;

R⁸ is H or C₁₋₄ alkyl which is optionally substituted by halo, CN, OR^{a9}, C(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, S(O)₂NR^{c9}R^{d9}, phenyl, C₃₋₇ cycloalkyl, a 5-6 membered heteroaryl moiety comprising carbon and 1, 2, or 3 heteroatoms independently selected from N, O and S, or a 4-7 membered heterocycloalkyl moiety comprising carbon and 1, 2, or 3 heteroatoms independently selected from N, O and S; wherein said phenyl, C₃₋₇ cycloalkyl, 5-6 membered heteroaryl, and 4-7 membered heterocycloalkyl groups of R⁸ are each optionally substituted with 1 or 2 R¹⁹;

R¹⁰ is selected from C₁₋₆ alkyl, C₂₋₆ alkenyl, C₂₋₆ alkynyl, C₁₋₆ haloalkyl, C₆₋₁₀ aryl, C₃₋₁₀ cycloalkyl, a 5-10 membered heteroaryl moiety comprising carbon and 1, 2, or 3 heteroatoms independently selected from N, O and S, and a 4-10 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl groups of R¹⁰ are each optionally substituted with 1, 2, 3, or 4 R^{10A};

R^{10A}, at each occurrence, is independently selected from halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, C₁₋₆ alkyl, C₂₋₆ alkenyl, C₂₋₆ alkynyl, C₁₋₆ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, phenyl, C₃₋₆ cycloalkyl, 5-6 membered heteroaryl, and 4-7 membered heterocycloalkyl group of R^{10A} are each optionally substituted with 1, 2, or 3 substituents independently selected from R¹⁹;

R^{a4}, R^{b4}, R^{c4}, and R^{d4}, at each occurrence, are independently selected from H, C₁₋₄ alkyl, C₂₋₄ alkenyl, C₂₋₄ alkynyl, C₁₋₄ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₆ cycloalkyl, 5-6 membered heteroaryl, and 4-7 membered heterocycloalkyl group of R^{a4}, R^{b4}, R^{c4}, and R^{d4} are each optionally substituted with 1, 2, or 3 substituents independently selected from R¹⁹;

alternatively, R^{e4} and R^{d4} together with the nitrogen atom to which they are attached form a 4-, 5-, 6-, or 7-membered heterocycloalkyl group which is optionally substituted with 1, 2 or 3 substituents independently selected from R¹⁹;

R^{e4} is H or C₁₋₄ alkyl;

R¹¹ is selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl, and C₂₋₆ alkynyl are each optionally substituted with 1, 2 or 3 substituents independently selected from R¹⁹;

alternatively, R¹⁰ and R¹¹ together with the carbon atom to which they are attached form a 3-, 4-, 5-, 6-, or 7-membered cycloalkyl group or a 4-, 5-, 6-, 7-, 8-, 9-, or 10-membered heterocycloalkyl group; wherein said 3-, 4-, 5-, 6-, or 7-membered cycloalkyl group and 4-, 5-, 6-, 7-, 8-, 9-, or 10-membered heterocycloalkyl group are each optionally substituted with 1, 2, 3 or 4 R^{10A};

R¹² is H or C₁₋₄ alkyl which is optionally substituted by R¹⁷;

R¹⁷, at each occurrence, is independently selected from halo, CN, NO₂, OR^{a7}, SR^{a7}, C(O)R^{b7}, C(O)NR^{c7}R^{d7}, C(O)OR^{a7}, OC(O)R^{b7}, OC(O)NR^{c7}R^{d7}, C(═NR^{e7})NR^{c7}R^{d7}, NR^{c7}C(═NR^{e7})NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)R^{b7}, NR^{c7}S(O)₂R^{b7}, NR^{c7}S(O)₂NR^{c7}R^{d7}, S(O)R^{b7}, S(O)NR^{c7}R^{d7}, S(O)₂R^{b7}, S(O)₂NR^{c7}R^{d7}, C₁₋₆ alkyl, C₂₋₆ alkenyl, C₂₋₆ alkynyl, C₁₋₆ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, phenyl, C₃₋₆ cycloalkyl, 5-6 membered heteroaryl, and 4-7 membered heterocycloalkyl groups of R¹⁷ are each optionally substituted with 1, 2, or 3 substituents independently selected from R¹⁹;

R^{a7}, R^{b7}, R^{c7}, and R^{d7}, at each occurrence, are independently selected from H, C₁₋₄ alkyl, C₂₋₄ alkenyl, C₂₋₄ alkynyl, C₁₋₄ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₆ cycloalkyl, 5-6 membered heteroaryl, and 4-7 membered heterocycloalkyl groups of R^{a7}, R^{b7}, R^{c7}, and R^{d7} are each optionally substituted with 1, 2, or 3 substituents independently selected from R¹⁹;

alternatively, R^{c7} and R^{d7} together with the nitrogen atom to which they are attached form a 4-, 5-, 6-, or 7-membered heterocycloalkyl group which is optionally substituted with 1, 2 or 3 substituents independently selected from R¹⁹;

R^{e7} is H or C₁₋₄ alkyl;

R¹⁹, at each occurrence, is independently selected from halo, CN, NO₂, OR^{a9}, SR^{a9}, C(O)R^{b9}, C(O)NR^{c9}R^{d9}, C(O)OR^{a9}, OC(O)R^{b9}, OC(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, S(O)₂NR^{c9}R^{d9}, C₁₋₄ alkyl, C₂₋₄ alkenyl, C₂₋₄ alkynyl, and C₁₋₄ haloalkyl;

R^{a9}, R^{c9}, and R^{d9}, at each occurrence, are independently selected from H and C₁₋₄ alkyl; and

R^{b9} is C₁₋₄ alkyl.

In some embodiments the present invention is directed to inhibitors of FGFR4 having Formula (II):

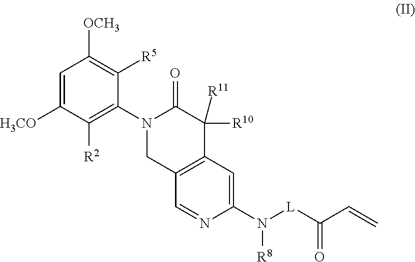

or a pharmaceutically acceptable salt thereof, wherein:

R² is F or Cl;

R⁵ is F or Cl;

L is a bond or *—CR^{7A}R^{7B} [symbol] CR^{7C}R^{7D}—NR^{7E}—, wherein the symbol * indicates the point of attachment to NR⁸ in Formula (I); wherein the symbol [symbol] represents a single or double bond; and wherein R^{7B} and R^{7D} are absent when [symbol] is a double bond;

R^{7A} is selected from H, C₁₋₄ alkyl, C₂₋₄ alkenyl, C₂₋₄ alkynyl, phenyl, C₃₋₇ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₇ cycloalkyl, 5-6 membered heteroaryl, and 4-7 membered heterocycloalkyl groups of R^{7A} are each optionally substituted with 1, 2, or 3 substituents independently selected from R¹⁷;

R^{7B} is H or C₁₋₄ alkyl optionally substituted with 1, 2, or 3 substituents independently selected from R¹⁷;

or R^{7A} and R^{7B} together with the carbon atom to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl and 4-7 membered heterocycloalkyl groups are each optionally substituted with 1, 2, or 3 substituents independently selected from R¹⁷;

R^{7C} is selected from H, C₁₋₄ alkyl, C₂₋₄ alkenyl, C₂₋₄ alkynyl, phenyl, C₃₋₇ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₇ cycloalkyl, 5-6 membered heteroaryl, and 4-7 membered heterocycloalkyl groups of R^{7C} are each optionally substituted with 1, 2, or 3 substituents independently selected from R¹⁷;

R^{7D} is H or C₁₋₄ alkyl optionally substituted with 1, 2, or 3 substituents independently selected from R¹⁷;

or R^{7C} and R^{7D} together with the carbon atom to which they are attached form a C₃₋₇ cycloalkyl or a 4-7 membered heterocycloalkyl moiety comprising carbon and 1, 2, or 3 heteroatoms independently selected from N, O and S; wherein said C₃₋₇ cycloalkyl and 4-7 membered heterocycloalkyl groups are each optionally substituted with 1, 2, or 3 substituents independently selected from R¹⁷;

R^{7E} is selected from H, C₁₋₄ alkyl, C₂₋₄ alkenyl, and C₂₋₄ alkynyl;

alternatively, when [symbol] is a double bond, R^{7A} and R^{7C} together with the carbon atoms to which they are attached form a phenyl group or a 5-6 membered heteroaryl moiety comprising carbon and 1, 2, or 3 heteroatoms independently selected from N, O and S, wherein said phenyl and 5-6 membered heteroaryl groups are each optionally substituted with 1, 2, or 3 substituents independently selected from R¹⁷;

alternatively, when [symbol] is a single bond, R^{7A} and R^{7C} together with the carbon atoms to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl group and 4-7 membered heterocycloalkyl group are each optionally substituted with 1, 2, or 3 substituents independently selected from R¹⁷;

alternatively, R^{7A} and R^{7E} together with the carbon and nitrogen atoms to which they are attached form a 5-6 membered heteroaryl moiety comprising carbon and 1, 2, or 3 nitrogen atoms, or a 4-7 membered heterocycloalkyl moiety comprising carbon and 1 or 2 nitrogen atoms; wherein said 5-6 membered heteroaryl and 4-7 membered heterocycloalkyl groups are each optionally substituted with 1, 2, or 3 substituents independently selected from R¹⁷;

alternatively, R^{7C} and R^{7E} together with the carbon and nitrogen atoms to which they are attached form a 5-6 membered heteroaryl moiety comprising carbon and 1, 2, or 3 nitrogen atoms, or a 4-7 membered heterocycloalkyl moiety comprising carbon and 1 or 2 nitrogen atoms; wherein said 5-6 membered heteroaryl and 4-7 membered heterocycloalkyl group are each optionally substituted with 1, 2, or 3 substituents independently selected from R¹⁷;

R⁸ is H or methyl;

R¹⁰ is selected from C₁₋₆ alkyl, C₂₋₆ alkenyl, C₂₋₆ alkynyl, C₁₋₆ haloalkyl, C₆₋₁₀ aryl, C₃₋₁₀ cycloalkyl, a 5-10 membered heteroaryl moiety comprising carbon and 1, 2, or 3 heteroatoms independently selected from N, O and S, and a 4-10 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl groups of R¹⁰ are each optionally substituted with 1, 2, 3, or 4 R^{10A};

R^{10A}, at each occurrence, is independently selected from halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4} S(O)₂NR^{c4}R^{d4}, C₁₋₆ alkyl, C₂₋₆ alkenyl, C₂₋₆ alkynyl, C₁₋₆ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, phenyl, C₃₋₆ cycloalkyl, 5-6 membered heteroaryl, and 4-7 membered heterocycloalkyl groups of R^{10a} are each optionally substituted with 1, 2, or 3 substituents independently selected from R¹⁹;

R^{a4}, R^{b4}, R^{c4}, and R^{d4}, at each occurrence, are independently selected from H, C₁₋₄ alkyl, C₂₋₄ alkenyl, C₂₋₄ alkynyl, C₁₋₄ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₆ cycloalkyl, 5-6 membered heteroaryl, and 4-7 membered heterocycloalkyl groups of R^{a4}, R^{b4}, R^{c4}, and R^{d4} are each optionally substituted with 1, 2, or 3 substituents independently selected from R¹⁹;

alternatively, R^{c4} and R^{d4} together with the nitrogen atom to which they are attached form a 4-, 5-, 6-, or 7-membered heterocycloalkyl group which is optionally substituted with 1, 2 or 3 substituents s independently selected from R¹⁹;

R^{e4} is H or C₁₋₄ alkyl;

R¹¹ is selected from C₁₋₆ alkyl, C₂₋₆ alkenyl, C₂₋₆ alkynyl, and C₁₋₆ haloalkyl;

alternatively, R¹⁰ and R¹¹ together with the carbon atom to which they are attached form a 3-, 4-, 5-, 6-, or 7-membered cycloalkyl group or a 4-, 5-, 6-, or 7-membered heterocycloalkyl group; wherein said 3-, 4-, 5-, 6-, or 7-membered cycloalkyl group and 4-, 5-, 6-, or 7-membered heterocycloalkyl group are each optionally substituted with 1, 2, 3 or 4 R^{10A};

R¹⁷, at each occurrence, is independently selected from OH, CN, amino, halo, C₁₋₆ alkyl, C₁₋₄ alkoxy, C₁₋₄ alkylthio, C₁₋₄ alkylamino, di(C₁₋₄ alkyl)amino, C₁₋₄ haloalkyl, and C₁₋₄ haloalkoxy;

R¹⁹, at each occurrence, is independently selected from halo, CN, NO₂, OR^{a9}, SR^{a9}, C(O)R^{b9}, C(O)NR^{c9}R^{d9}, C(O)OR^{a9}, OC(O)R^{b9}, OC(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, S(O)₂NR^{c9}R^{d9}, C₁₋₄ alkyl, C₂₋₄ alkenyl, C₂₋₄ alkynyl, and C₁₋₄ haloalkyl;

R^{a9}, R^{c9}, and R^{d9}, at each occurrence, are independently selected from H and C₁₋₄ alkyl; and

R^{b9} is C₁₋₄ alkyl.

In some embodiments, the compounds of the invention have Formula (V):

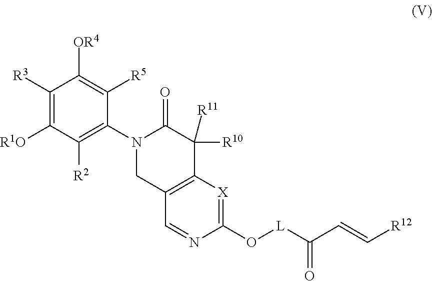

wherein:

L is a bond or *—CR^{7A}R^{7B} [symbol] CR^{7C}R^{7D}—NR^{7E}—, wherein the symbol * indicates the point of attachment to —O— in Formula (V); wherein the symbol [symbol] represents a single or double bond; and wherein R^{7B} and R^{7D} are absent when [symbol] is a double bond.

In some embodiments, X is N.

In some embodiments, X is CR⁶.

In some embodiments, R⁶ is H, halo, CN, or C₁₋₆ alkyl. In some embodiments, R⁶ is H. In some embodiments, R⁶ is C₁₋₆ alkyl. In some embodiments, R⁶ is methyl. In some embodiments, R⁶ is halo. In some embodiments, R⁶ is CN.

In some embodiments, R¹ is C₁₋₃ alkyl. In some embodiments, R¹ is methyl.

In some embodiments, R² is halo. In some embodiments, R² is fluoro. In some embodiments, R² is chloro.

In some embodiments, R³ is H.

In some embodiments, R⁴ is C₁₋₃ alkyl. In some embodiments, R⁴ is methyl.

In some embodiments, R⁵ is halo. In some embodiments, R⁵ is fluoro. In some embodiments, R⁵ is chloro.

In some embodiments, R² is fluoro and R⁵ is fluoro. In some embodiments, R² is chloro and R⁵ is chloro.

In some embodiments, R¹ is C₁₋₃ alkyl; R² is halo; R³ is H; R⁴ is C₁₋₃ alkyl; and R⁵ is halo.

In some embodiments, R¹ is C₁₋₃ alkyl; R² is F; R³ is H; R⁴ is C₁₋₃ alkyl; and R⁵ is F.

In some embodiments, R¹ is methyl; R² is F; R³ is H; R⁴ is methyl; and R⁵ is F.

In some embodiments, R¹ is C₁₋₃ alkyl; R² is Cl; R³ is H; R⁴ is C₁₋₃ alkyl; and R⁵ is Cl.

In some embodiments, R¹ is methyl; R² is Cl; R³ is H; R⁴ is methyl; and R⁵ is Cl.

In some embodiments, R¹⁰ is C₁₋₆ alkyl. In some embodiments, R¹⁰ is methyl.

In some embodiments, R¹¹ is C₁₋₆ alkyl. In some embodiments, R¹¹ is methyl.

In some embodiments, R¹⁰ and R¹¹ are each C₁₋₆ alkyl. In some embodiments, R¹⁰ and R¹¹ are each methyl.

In some embodiments, R¹⁰ and R¹¹ together with the carbon atom to which they are attached form a 3-, 4-, 5-, 6-, or 7-membered cycloalkyl group. In some embodiments, R¹⁰ and R¹¹ together with the carbon atom to which they are attached form a 3-, 4-, 5-, or 6-membered cycloalkyl group. In some embodiments, R¹⁰ and R¹¹ together with the carbon atom to which they are attached form a 3-, 4-, or 5-membered cycloalkyl group.

In some embodiments, R¹⁰ and R¹¹ together with the carbon atom to which they are attached form a cyclopropyl group. In some embodiments, R¹⁰ and R¹¹ together with the carbon atom to which they are attached form a cyclobutyl group. In some embodiments, R¹⁰ and R¹¹ together with the carbon atom to which they are attached form a cyclopentyl group. In some embodiments, R¹⁰ and R¹¹ together with the carbon atom to which they are attached form a cyclohexyl group. In some embodiments, R¹⁰ and R¹¹ together with the carbon atom to which they are attached form a cycloheptyl group.

In some embodiments, R¹⁰ and R¹¹ together with the carbon atom to which they are attached form a cyclopropyl group optionally substituted by 1 or 2 R^{10A}. In some embodiments, R¹⁰ and R¹¹ together with the carbon atom to which they are attached form a cyclobutyl group optionally substituted by 1 or 2 R^{10A}. In some embodiments, R¹⁰ and R¹¹ together with the carbon atom to which they are attached form a cyclopentyl group optionally substituted by 1 or 2 R^{10A}. In some embodiments, R¹⁰ and R¹¹ together with the carbon atom to which they are attached form a cyclohexyl group optionally substituted by 1 or 2 R^{10A}.

In some embodiments, R¹⁰ and R¹¹ together with the carbon atom to which they are attached form 4-, 5-, 6-, or 7-membered heterocycloalkyl group.

In some embodiments, R¹⁰ and R¹¹ together with the carbon atom to which they are attached form a tetrahydropyranyl group, a tetrahydrofuranyl group, tetrahydrothiophene group, a pyrrolidinyl group, or a piperidinyl group. In some embodiments, R¹⁰ and R¹¹ together with the carbon atom to which they are attached form a tetrahydropyranyl group. In some embodiments, R¹⁰ and R¹¹ together with the carbon atom to which they are attached form a tetrahydropyranyl group optionally substituted by 1 or 2 R^{10A}. In some embodiments, R¹⁰ and R¹¹ together with the carbon atom to which they are attached form a tetrahydrofuranyl group. In some embodiments, R¹⁰ and R¹¹ together with the carbon atom to which they are attached form a tetrahydrofuranyl group optionally substituted by R^{10A}. In some embodiments, R¹⁰ and R¹¹ together with the carbon atom to which they are attached form an azetidinyl group. In some embodiments, R¹⁰ and R¹¹ together with the carbon atom to which they are attached form an azetidinyl group optionally substituted by R^{10A}.

In some embodiments, L is a bond.

In some embodiments, L is —(C₂₋₆ alkylene)-NH—, —(C₂₋₆ alkenylene)-NH—, —(C₂₋₆ alkynylene)-NH—, —(C₆₋₁₀ aryl)-NH—, —(C₃₋₁₀ cycloalkyl)-NH—, a (5-10 membered heteroaryl)-NH—, or a (4-10 membered heterocycloalkyl)-NH— group, wherein each C₂₋₆ alkylene, C₂₋₆ alkenylene, C₂₋₆ alkynylene, C₆-10 aryl, C₃₋₁₀ cycloalkyl, 5-10 membered heteroaryl, or 4-10 membered heterocycloalkyl group is optionally substituted with 0, 1 or 2 R¹⁷.

In some embodiments, L is —(C₂₋₆ alkylene)-NH—. In some embodiments, L is a —(CH₂ CH₂)—NH—. In some embodiments, L is —(C₂₋₆ alkenylene)-NH—.

In some embodiments, L is a —(C₆₋₁₀ aryl)-NH— group optionally substituted with 0, 1 or 2 R¹⁷. In some embodiments, L is a -(phenyl)-NH— group optionally substituted with 0, 1 or 2 R¹⁷.

In some embodiments, L is a -(5-10 membered heteroaryl)-NH— group optionally substituted with 0, 1 or 2 R¹⁷. In some embodiments, L is a -(pyridyl)-NH— group optionally substituted with 0, 1 or 2 R¹⁷. In some embodiments, L is a -(pyrimidinyl)-NH— group optionally substituted with 0, 1 or 2 R¹⁷. In some embodiments, L is an -(imidazolyl)-NH-group optionally substituted with 0 or 1 R¹⁷. In some embodiments, L is an -(pyrazolyl)-NH-group optionally substituted with 0 or 1 R¹⁷.

In some embodiments, L is a -(4-10 membered heterocycloalkyl)-NH— group optionally substituted with 0, 1 or 2 R¹⁷. In some embodiments, L is a -(4-6 membered heterocycloalkyl)-NH-group optionally substituted with 0, 1 or 2 R¹⁷. In some embodiments, L is a -(tetrahydrofuranyl)-NH— group optionally substituted with 0, 1 or 2 R¹⁷. In some embodiments, L is a -(tetrahydropyranyl)-NH— group optionally substituted with 0, 1 or 2 R¹⁷. In some embodiments, L is a -(pyrrolidinyl)-NH— group optionally substituted with 0, 1 or 2 R¹⁷. In some embodiments, L is a -(piperidinyl)-NH— group optionally substituted with 0, 1 or 2 R¹⁷.

In some embodiments, L is a —(C₃₋₁₀ cycloalkyl)-NH— group optionally substituted with 0, 1 or 2 R¹⁷. In some embodiments, L is a —(C₃₋₇ cycloalkyl)-NH— group optionally substituted with 0, 1 or 2 R¹⁷. In some embodiments, L is a -(cyclopropyl)-NH— group optionally substituted with 0, 1 or 2 R¹⁷. In some embodiments, L is a -(cyclobutyl)-NH— group optionally substituted with 0, 1 or 2 R¹⁷. In some embodiments, L is a -(cyclopentyl)-NH— group optionally substituted with 0, 1 or 2 R¹⁷. In some embodiments, L is a -(cyclohexyl)-NH— group optionally substituted with 0, 1 or 2 R¹⁷.

In some embodiments, L is

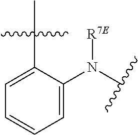

wherein, R^{7E} is H or C₁₋₄ alkyl, and the phenyl group is optionally substituted with 0, 1 or 2 R¹⁷, wherein R¹⁷, at each occurrence, is independently selected from OH, CN, amino, halo, C₁₋₆ alkyl, C₁₋₄ alkoxy, C₁₋₄ alkylthio, C₁₋₄ alkylamino, di(C₁₋₄ alkyl)amino, C₁₋₄ haloalkyl, and C₁₋₄ haloalkoxy. In a preferred embodiment, R^{7E} is H.

In some embodiments, L is

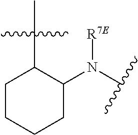

wherein, R^{7E} is H or C₁₋₄ alkyl, and the cyclohexyl group is optionally substituted with 0, 1 or 2 R¹⁷, wherein R¹⁷, at each occurrence, is independently selected from OH, CN, amino, halo, C₁₋₆ alkyl, C₁₋₄ alkoxy, C₁₋₄ alkylthio, C₁₋₄ alkylamino, di(C₁₋₄ alkyl)amino, C₁₋₄ haloalkyl, and C₁₋₄ haloalkoxy. In a preferred embodiment, R^{7E} is H.

In some embodiments, L is

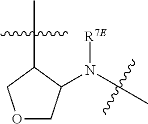

wherein, R^{7E} is H or C₁₋₄ alkyl, and the tetrahydrofuranyl group is optionally substituted with 0, 1 or 2 R¹⁷, wherein R¹⁷, at each occurrence, is independently selected from OH, CN, amino, halo, C₁₋₆ alkyl, C₁₋₄ alkoxy, C₁₋₄ alkylthio, C₁₋₄ alkylamino, di(C₁₋₄ alkyl)amino, C₁₋₄ haloalkyl, and C₁₋₄ haloalkoxy. In a preferred embodiment, R^{7E} is H.

In some embodiments, L is

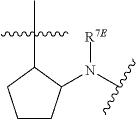

wherein, R^{7E} is H or C₁₋₄ alkyl, and the tetrahydrofuranyl group is optionally substituted with 0, 1 or 2 R¹⁷, wherein R¹⁷, at each occurrence, is independently selected from OH, CN, amino, halo, C₁₋₆ alkyl, C₁₋₄ alkoxy, C₁₋₄ alkylthio, C₁₋₄ alkylamino, di(C₁₋₄ alkyl)amino, C₁₋₄ haloalkyl, and C₁₋₄ haloalkoxy. In a preferred embodiment, R^{7E} is H.

In some embodiments, L is selected from:

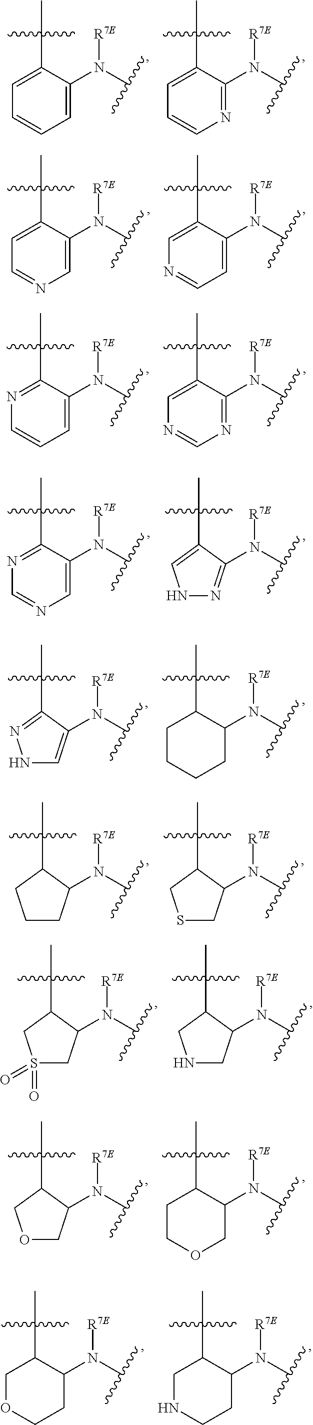

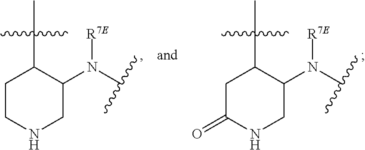

R^{7E} is H or C₁₋₄ alkyl, and the phenyl, pyridinyl, pyrimidinyl, pyrazolyl, cyclohexyl, cyclopentyl, tetrahydrothiophenyl, tetrahydrothiophene-1,1-dioxide (sulfolanyl), pyrrolidinyl, tetrahydrofuranyl, tetrahydropyranyl, piperidinyl, and piperidin-2-one group is optionally substituted with 0, 1 or 2 R¹⁷, wherein R¹⁷, at each occurrence, is independently selected from OH, CN, amino, halo, C₁₋₆ alkyl, C₁₋₄ alkoxy, C₁₋₄ alkylthio, C₁₋₄ alkylamino, di(C₁₋₄ alkyl)amino, C₁₋₄ haloalkyl, and C₁₋₄ haloalkoxy. In a preferred embodiment, R^{7E} is H.

In some embodiments, L is *—CR^{7A}R^{7B} [symbol] CR^{7C}R^{7D}—NR^{7E}—, wherein the symbol * indicates the point of attachment to NR⁸ in Formula (I); wherein the symbol [symbol] represents a single or double bond; and wherein R^{7B} and R^{7D} are absent when [symbol] is a double bond.

In some embodiments:

L is *—CR^{7A}R^{7B} [symbol] CR^{7C}R^{7D}—NR^{7E}—, wherein the symbol * indicates the point of attachment to NR⁸ in Formula (I); wherein the symbol [symbol] represents a single or double bond; and wherein R^{7B} and R^{7D} are absent when [symbol] is a double bond;

R^{7A} is selected from H, C₁₋₄ alkyl, C₂₋₄ alkenyl, and C₂₋₄ alkynyl;

R^{7C} is selected from H, C₁₋₄ alkyl, C₂₋₄ alkenyl, and C₂₋₄ alkynyl;

alternatively, when [symbol] is a double bond, R^{7A} and R^{7C} together with the carbon atoms to which they are attached form a phenyl group or a 5-6 membered heteroaryl moiety comprising carbon and 1, 2, or 3 heteroatoms independently selected from N, O and S, wherein said phenyl and 5-6 membered heteroaryl groups are each optionally substituted with 1, 2, or 3 substituents independently selected from R¹⁷;

alternatively, when [symbol] is a single bond, R^{7A} and R^{7C} together with the carbon atoms to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl group and 4-7 membered heterocycloalkyl group are each optionally substituted with 1, 2, or 3 substituents independently selected from R¹⁷;

R^{7E} is selected from H or C₁₋₄ alkyl;

R¹⁷, at each occurrence, is independently selected from OH, CN, amino, halo, C₁₋₆ alkyl, C₁₋₄ alkoxy, C₁₋₄ alkylthio, C₁₋₄ alkylamino, di(C₁₋₄ alkyl)amino, C₁₋₄ haloalkyl, and C₁₋₄ haloalkoxy; and

R⁸ is H.

In some embodiments:

L is *—CR^{7A}R^{7B} [symbol] CR^{7C}R^{7D}—NR^{7E}—, wherein the symbol * indicates the point of attachment to NR⁸ in Formula (I), and wherein the symbol [symbol] represents a single bond;

R^{7A} and R^{7C} together with the carbon atoms to which they are attached form a C₃₋₇ cycloalkyl optionally substituted with 1, 2, or 3 substituents independently from R¹⁷;

R^{7E} is selected from H or methyl;

R¹⁷, at each occurrence, is independently selected from OH, CN, amino, halo, C₁₋₆ alkyl, C₁₋₄ alkoxy, C₁₋₄ alkylthio, C₁₋₄ alkylamino, di(C₁₋₄ alkyl)amino, C₁₋₄ haloalkyl, and C₁₋₄ haloalkoxy; and

R⁸ is H.

In some embodiments, R^{7A} and R^{7C} together with the carbon atoms to which they are attached form a cyclohexyl group.

In some embodiments:

L is *—CR^{7A}R^{7B} [symbol] CR^{7C}R^{7D}—NR^{7E}—, wherein the symbol * indicates the point of attachment to NR⁸ in Formula (I), wherein the symbol [symbol] represents a double bond, and wherein R^{7B} and R^{7D} are absent;

R^{7A} and R^{7C} together with the carbon atoms to which they are attached form a phenyl optionally substituted with 1, 2, or 3 substituents independently from R¹⁷;

R^{7E} is selected from H or methyl;

R¹⁷, at each occurrence, is independently selected from OH, CN, amino, halo, C₁₋₆ alkyl, C₁₋₄ alkoxy, C₁₋₄ alkylthio, C₁₋₄ alkylamino, di(C₁₋₄ alkyl)amino, C₁₋₄ haloalkyl, and C₁₋₄ haloalkoxy; and

R⁸ is H.

In some embodiments:

L is *—CR^{7A}R^{7B} [symbol] CR^{7C}R^{7D}—NR^{7E}—, wherein the symbol * indicates the point of attachment to NR⁸ in Formula (I), and wherein the symbol [symbol] represents a single bond;

R^{7A} and R^{7C} together with the carbon atoms to which they are attached form a 4-7 membered heterocycloalkyl moiety comprising carbon and 1, 2, or 3 heteroatoms independently from N, O and S, optionally substituted with 1, 2, or 3 substituents independently selected from R¹⁷;

R^{7E} is selected from H or methyl;

R¹⁷, at each occurrence, is independently selected from OH, CN, amino, halo, C₁₋₆ alkyl, C₁₋₄ alkoxy, C₁₋₄ alkylthio, C₁₋₄ alkylamino, di(C₁₋₄ alkyl)amino, C₁₋₄ haloalkyl, and C₁₋₄ haloalkoxy; and

R⁸ is H.

In some embodiments, L is

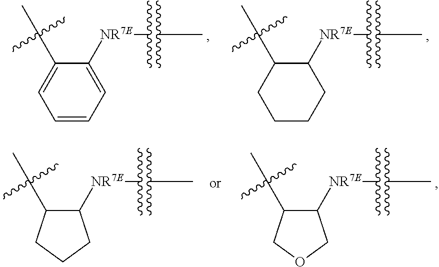

each of which is optionally substituted with from 1 to 3 R¹⁷ groups, wherein the single wavy lines indicates the point of the attachment to Y, O, or NR⁸ in formula (I′), (I), (II) or (V) and the double wavy lines indicate the point of attachment to the carbonyl group of the —C(O)CH═CHR¹² moiety in formula (I′), (I), (II) or (V).

In some embodiments, R^{7A} and R^{7C} together with the carbon atoms to which they are attached form a tetrahydrofuranyl moiety.

In some embodiments, R^{7A} is H. In some embodiments, R^{7A} is C₁₋₄ alkyl.

In some embodiments, R^{7B} is H. In some embodiments, R^{7B} is C₁₋₄ alkyl.

In some embodiments, R^{7C} is H. In some embodiments, R^{7C} is C₁₋₄ alkyl.

In some embodiments, R^{7D} is H. In some embodiments, R^{7D} is C₁₋₄ alkyl.

In some embodiments, R^{7E} is H. In some embodiments, R^{7E} is methyl.

In some embodiments, R¹⁷ is methyl.

In some embodiments, R⁸ is H or C₁₋₄ alkyl. In some embodiments, R⁸ is H or methyl. In some embodiments, R⁸ is H. In some embodiments, R⁸ is methyl.

In some embodiments, R¹² is H or C₁₋₄ alkyl which is optionally substituted by R¹⁹; wherein R¹⁹, at each occurrence, is independently selected from halo, CN, NO₂, OR^{a9}, SR^{a9}, C(O)R^{b9}, C(O)NR^{c9}R^{d9}, C(O)OR^{a9}, OC(O)R^{b9}, OC(O)NR^{c9}R^{d9}, NR^{c9}R^{d9}, NR^{c9}C(O)R^{b9} NR^{c9}C(O)OR^{a9}, NR^{c9}C(O)NR^{c9}R^{d9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, S(O)₂NR^{c9}R^{d9}, C₁₋₄ alkyl, C₂₋₄ alkenyl, C₂₋₄ alkynyl, and C₁₋₄ haloalkyl; R^{a9}, R^{c9}, and R^{d9}, at each occurrence, are independently selected from H and C₁₋₄ alkyl; and R^{b9} is C₁₋₄ alkyl.

In some embodiments, R¹² is H or C₁₋₄ alkyl. In some embodiments, R¹² is C₁₋₄ alkyl. In some embodiments, R¹² is C₁₋₄ alkyl substituted by —N(CH₃)₂. In some embodiments, R¹² is —CH₂—N(CH₃)₂. In some embodiments, R¹² is methyl. In some embodiments, R¹² is C₁₋₄ alkyl substituted by piperidin-1-yl. In some embodiments, R¹² is —CH₂(piperidin-1-yl).

In some embodiments, R¹² is H.

In some embodiments, the present invention is an inhibitor of FGFR4 which is a compound having Formula (III):

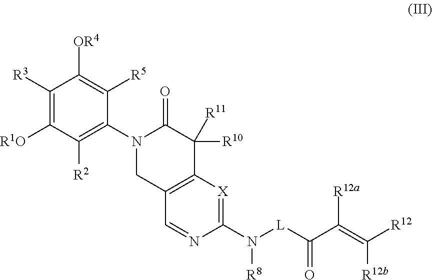

or a pharmaceutically acceptable salt thereof, wherein X, L, R¹, R², R³, R⁴, R⁵, R⁸, R¹⁰, R¹¹, and R¹² are as defined herein; R^{12a} is H, halo, C₁₋₃ alkyl, or C₁₋₃ haloalkyl; and R^{12b} is H, halo, C₁₋₃ alkyl, or C₁₋₃ haloalkyl.

In some embodiments the present invention is an inhibitor of FGFR4 which is a compound having Formula (IV):

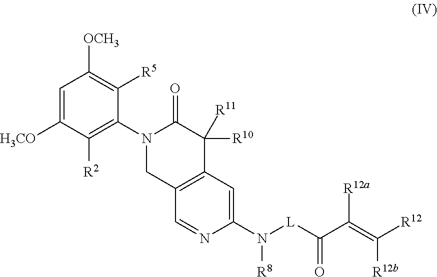

or a pharmaceutically acceptable salt thereof, wherein L, R², R⁵, R⁸, R¹⁰, R¹¹, and R¹² are as defined herein; R^{12a} is H, halo, C₁₋₃ alkyl, or C₁₋₃ haloalkyl; and R^{12b} is H, halo, C₁₋₃ alkyl, or C₁₋₃ haloalkyl.

In some embodiments, R^{12a} is H, F, methyl, or trifluoromethyl.

In some embodiments, R^{12b} is H, F, methyl, or trifluoromethyl.

In some embodiments the present invention is an inhibitor of FGFR4 which is a compound having Formula (Va):

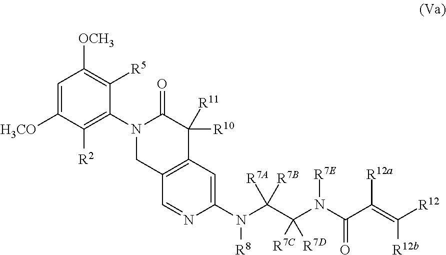

or a pharmaceutically acceptable salt thereof, wherein L, R², R⁵, R⁸, R¹⁰, R¹¹, and R¹² are as defined herein; R^{12a} is H, halo, C₁₋₃ alkyl, or C₁₋₃ haloalkyl; and R^{12b} is H, halo, C₁₋₃ alkyl, or C₁₋₃ haloalkyl.

In some embodiments, R^{12a} is H, F, methyl, or trifluoromethyl.

In some embodiments, R^{12b} is H, F, methyl, or trifluoromethyl.

In some embodiments the present invention is an inhibitor of FGFR4 which is a compound having Formula (Vb):

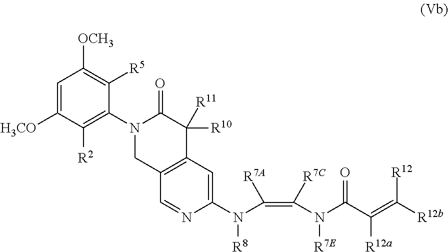

or a pharmaceutically acceptable salt thereof, wherein L, R², R⁵, R⁸, R¹⁰, R¹¹, and R¹² are as defined herein; R^{12a} is H, halo, C₁₋₃ alkyl, or C₁₋₃ haloalkyl; and R^{12b} is H, halo, C₁₋₃ alkyl, or C₁₋₃ haloalkyl.

In some embodiments, R^{12a} is H, F, methyl, or trifluoromethyl.

In some embodiments, R^{12b} is H, F, methyl, or trifluoromethyl.

In some embodiments the present invention is an inhibitor of FGFR4 which is a compound having Formula (Vc):

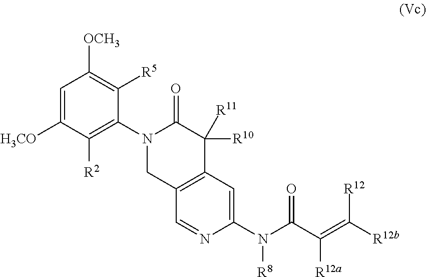

or a pharmaceutically acceptable salt thereof, wherein L, R², R⁵, R⁸, R¹⁰, R¹¹, and R¹² are as defined herein; R^{12a} is H, halo, C₁₋₃ alkyl, or C₁₋₃ haloalkyl; and R^{12b} is H, halo, C₁₋₃ alkyl, or C₁₋₃ haloalkyl.

In some embodiments, R^{12a} is H, F, methyl, or trifluoromethyl.

In some embodiments, R^{12b} is H, F, methyl, or trifluoromethyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the phrase “optionally substituted” means unsubstituted or substituted.

As used herein, the term “substituted” means that a hydrogen atom is replaced by a non-hydrogen group. It is to be understood that substitution at a given atom is limited by valency.

As used herein, the term “Cᵢ₋ⱼ,” where i and j are integers, employed in combination with a chemical group, designates a range of the number of carbon atoms in the chemical group with i-j defining the range. For example, C₁₋₆ alkyl refers to an alkyl group having 1, 2, 3, 4, 5, or 6 carbon atoms.

As used herein, the term “alkyl,” employed alone or in combination with other terms, refers to a saturated hydrocarbon group that may be straight-chain or branched. In some embodiments, the alkyl group contains 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, and the like. In some embodiments, the alkyl group is methyl, ethyl, or propyl.

As used herein, “alkenyl,” employed alone or in combination with other terms, refers to an alkyl group having one or more carbon-carbon double bonds. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, “alkynyl,” employed alone or in combination with other terms, refers to an alkyl group having one or more carbon-carbon triple bonds. In some embodiments, the alkynyl moiety contains 2 to 6 or 2 to 4 carbon atoms. Example alkynyl groups include, but are not limited to, ethynyl, propyn-1-yl, propyn-2-yl, and the like.

As used herein, “halo” or “halogen”, employed alone or in combination with other terms, includes fluoro, chloro, bromo, and iodo. In some embodiments, halo is F or Cl. In some embodiments, halo is F.

As used herein, the term “haloalkyl,” employed alone or in combination with other terms, refers to an alkyl group having up to the full valency of halogen atom substituents, which may either be the same or different. In some embodiments, the halogen atoms are fluoro atoms. In some embodiments, the alkyl group has 1 to 6, 1 to 4, or 1 to 3 carbon atoms. Example haloalkyl groups include CF₃, C₂F₅, CHF₂, CCl₃, CHCl₂, C₂Cl₅, and the like.

As used herein, the term “alkoxy,” employed alone or in combination with other terms, refers to a group of formula —O-alkyl. In some embodiments, the alkyl group has 1 to 6, 1 to 4, or 1 to 3 carbon atoms. Example alkoxy groups include methoxy, ethoxy, propoxy (e.g., n-propoxy and isopropoxy), t-butoxy, and the like. In some embodiments, alkoxy is methoxy.

As used herein, “haloalkoxy,” employed alone or in combination with other terms, refers to a group of formula —O-(haloalkyl). In some embodiments, the alkyl group has 1 to 6, 1 to 4, or 1 to 3 carbon atoms. An example haloalkoxy group is —OCF₃.

As used herein, “amino,” employed alone or in combination with other terms, refers to NH₂.

As used herein, the term “alkylamino,” employed alone or in combination with other terms, refers to a group of formula —NH(alkyl). In some embodiments, the alkylamino group has 1 to 6 or 1 to 4 carbon atoms. Example alkylamino groups include methylamino, ethylamino, propylamino (e.g., n-propylamino and isopropylamino), and the like.

As used herein, the term “dialkylamino,” employed alone or in combination with other terms, refers to a group of formula —N(alkyl)₂. Example dialkylamino groups include dimethylamino, diethylamino, dipropylamino (e.g., di(n-propyl)amino and di(isopropyl)amino), and the like. In some embodiments, each alkyl group independently has 1 to 6 or 1 to 4 carbon atoms.

As used herein, the term “alkylthio,” employed alone or in combination with other terms, refers to a group of formula —S-alkyl. In some embodiments, the alkyl group has 1 to 6 or 1 to 4 carbon atoms.

As used herein, the term “cycloalkyl,” employed alone or in combination with other terms, refers to a non-aromatic cyclic hydrocarbon including cyclized alkyl and alkenyl groups.

Cycloalkyl groups can include mono- or polycyclic (e.g., having 2, 3, or 4 fused, bridged, or spiro rings) ring systems. Also included in the definition of cycloalkyl are moieties that have one or more aromatic rings (e.g., aryl or heteroaryl rings) fused (i.e., having a bond in common with) to the cycloalkyl ring, for example, benzo derivatives of cyclopentane, cyclohexene, cyclohexane, and the like, or pyrido derivatives of cyclopentane or cyclohexane. Ring-forming carbon atoms of a cycloalkyl group can be optionally substituted by oxo. Cycloalkyl groups also include cycloalkylidenes. The term “cycloalkyl” also includes bridgehead cycloalkyl groups (e.g., non-aromatic cyclic hydrocarbon moieties containing at least one bridgehead carbon, such as admantan-1-yl) and spirocycloalkyl groups (e.g., non-aromatic hydrocarbon moieties containing at least two rings fused at a single carbon atom, such as spiro[2.5]octane and the like). In some embodiments, the cycloalkyl group has 3 to 10 ring members, or 3 to 7 ring members, or 3 to 6 ring members. In some embodiments, the cycloalkyl group is monocyclic or bicyclic. In some embodiments, the cycloalkyl group is monocyclic. In some embodiments, the cycloalkyl group is a C₃₋₇ monocyclic cycloalkyl group. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, tetrahydronaphthalenyl, octahydronaphthalenyl, indanyl, and the like. In some embodiments, the cycloalkyl group is cyclopropyl, cyclobutyl, cyclopentyl, or cyclohexyl.

As used herein, the term “heterocycloalkyl,” employed alone or in combination with other terms, refers to a non-aromatic ring or ring system, which may optionally contain one or more alkenylene or alkynylene groups as part of the ring structure, which has at least one heteroatom ring member independently selected from nitrogen, sulfur, oxygen, and phosphorus. Heterocycloalkyl groups can include mono- or polycyclic (e.g., having 2, 3 or 4 fused, bridged, or spiro rings) ring systems. In some embodiments, the heterocycloalkyl group is a monocyclic or bicyclic group having 1, 2, 3, or 4 heteroatoms independently selected from nitrogen, sulfur and oxygen. Also included in the definition of heterocycloalkyl are moieties that have one or more aromatic rings (e.g., aryl or heteroaryl rings) fused (i.e., having a bond in common with) to the non-aromatic heterocycloalkyl ring, for example, 1,2,3,4-tetrahydro-quinoline and the like. Heterocycloalkyl groups can also include bridgehead heterocycloalkyl groups (e.g., a heterocycloalkyl moiety containing at least one bridgehead atom, such as azaadmantan-1-yl and the like) and spiroheterocycloalkyl groups (e.g., a heterocycloalkyl moiety containing at least two rings fused at a single atom, such as [1,4-dioxa-8-aza-spiro[4.5]decan-N-yl] and the like). In some embodiments, the heterocycloalkyl group has 3 to 10 ring-forming atoms, 4 to 10 ring-forming atoms, or 3 to 8 ring forming atoms. In some embodiments, the heterocycloalkyl group has 1 to 5 heteroatoms, 1 to 4 heteroatoms, 1 to 3 heteroatoms, or 1 to 2 heteroatoms. The carbon atoms or heteroatoms in the ring(s) of the heterocycloalkyl group can be oxidized to form a carbonyl, an N-oxide, or a sulfonyl group (or other oxidized linkage) or a nitrogen atom can be quaternized. In some embodiments, the heterocycloalkyl portion is a C₂₋₇ monocyclic heterocycloalkyl group. In some embodiments, the heterocycloalkyl group is a morpholine ring, pyrrolidine ring, piperazine ring, piperidine ring, dihydropyran ring, tetrahydropyran ring, tetrahyropyridine, azetidine ring, or tetrahydrofuran ring.

As used herein, the term “aryl,” employed alone or in combination with other terms, refers to a monocyclic or polycyclic (e.g., having 2 fused rings) aromatic hydrocarbon moiety, such as, but not limited to, phenyl, 1-naphthyl, 2-naphthyl, and the like. In some embodiments, aryl groups have from 6 to 10 carbon atoms or 6 carbon atoms. In some embodiments, the aryl group is a monocyclic or bicyclic group. In some embodiments, the aryl group is phenyl or naphthyl.

As used herein, the term “heteroaryl,” employed alone or in combination with other terms, refers to a monocyclic or polycyclic (e.g., having 2 or 3 fused rings) aromatic hydrocarbon moiety, having one or more heteroatom ring members independently selected from nitrogen, sulfur and oxygen. In some embodiments, the heteroaryl group is a monocyclic or bicyclic group having 1, 2, 3, or 4 heteroatoms independently selected from nitrogen, sulfur and oxygen. Example heteroaryl groups include, but are not limited to, pyridyl, pyrimidinyl, pyrazinyl, pyridazinyl, triazinyl, furyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, pyrrolyl, azolyl, quinolinyl, isoquinolinyl, benzisoxazolyl, imidazo[1,2-b]thiazolyl or the like. The carbon atoms or heteroatoms in the ring(s) of the heteroaryl group can be oxidized to form a carbonyl, an N-oxide, or a sulfonyl group (or other oxidized linkage) or a nitrogen atom can be quaternized, provided the aromatic nature of the ring is preserved. In one embodiment the heteroaryl group is a 5 to 10 membered heteroaryl group. In another embodiment the heteroaryl group is a 5 to 6 membered heteroaryl group.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by methods known in the art. An example method includes fractional recrystallizaion using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Other resolving agents suitable for fractional crystallization methods include stereoisomerically pure forms of methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine-imine pairs, and annular forms where a proton can occupy two or more positions of a heterocyclic system, for example, 1H- and 3H-imidazole, 1H-, 2H— and 4H-1,2,4-triazole, 1H- and 2H-isoindole, and 1H- and 2H-pyrazole. Tautomeric forms can be in equilibrium or sterically locked into one form by appropriate substitution.

Compounds of the invention also include all isotopes of atoms occurring in the intermediates or final compounds. Isotopes include those atoms having the same atomic number but different mass numbers. For example, isotopes of hydrogen include tritium and deuterium.

The term, “compound,” as used herein is meant to include all stereoisomers, geometric iosomers, tautomers, and isotopes of the structures depicted.

All compounds, and pharmaceutically acceptable salts thereof, can be found together with other substances such as water and solvents (e.g., in the form of hydrates and solvates) or can be isolated.

In some embodiments, the compounds of the invention, or salts thereof, are substantially isolated. By “substantially isolated” is meant that the compound is at least partially or substantially separated from the environment in which it was formed or detected. Partial separation can include, for example, a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

The following abbreviations may be used herein: AcOH (acetic acid); Ac₂O (acetic anhydride); aq. (aqueous); atm. (atmosphere(s)); Boc (t-butoxycarbonyl); br (broad); Cbz (carboxybenzyl); calc. (calculated); d (doublet); dd (doublet of doublets); DCM (dichloromethane); DEAD (diethyl azodicarboxylate); DIAD (N,N′-diisopropyl azidodicarboxylate); DIPEA (N,N-diisopropylethylamine); DMF (N,N-dimethylformamide); Et (ethyl); EtOAc (ethyl acetate); g (gram(s)); h (hour(s)); HATU (N,N,N′,N′-tetramethyl-O-(7-azabenzotriazol-1-yl)uronium hexafluorophosphate); HCl (hydrochloric acid); HPLC (high performance liquid chromatography); Hz (hertz); J (coupling constant); LCMS (liquid chromatography—mass spectrometry); m (multiplet); M (molar); mCPBA (3-chloroperoxybenzoic acid); MgSO₄ (magnesium sulfate); MS (Mass spectrometry); Me (methyl); MeCN (acetonitrile); MeOH (methanol); mg (milligram(s)); min. (minutes(s)); mL (milliliter(s)); mmol (millimole(s)); N (normal); NaHCO₃ (sodium bicarbonate); NaOH (sodium hydroxide); Na₂SO₄ (sodium sulfate); NH₄Cl (ammonium chloride); NH₄OH (ammonium hydroxide); nM (nanomolar); NMR (nuclear magnetic resonance spectroscopy); OTf (trifluoromethanesulfonate); Pd (palladium); Ph (phenyl); pM (picomolar); PMB (para-methoxybenzyl), POCl₃ (phosphoryl chloride); RP-HPLC (reverse phase high performance liquid chromatography); s (singlet); t (triplet or tertiary); TBS (tert-butyldimethylsilyl); tert (tertiary); tt (triplet of triplets); t-Bu (tert-butyl); TFA (trifluoroacetic acid); THF (tetrahydrofuran); μg (microgram(s)); μL (microliter(s)); μM (micromolar); wt % (weight percent)

### Synthesis

Compounds of the invention, including salts thereof, can be prepared using known organic synthesis techniques and according to various possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P.G.M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature,” “room temperature,” and “r.t.”, as used herein, are understood in the art, and refer generally to a temperature, e.g. a reaction temperature, that is about the temperature of the room in which the reaction is carried out, for example, a temperature from about 20° C. to about 30° C.

Compounds of the invention can be prepared by one skilled in the art according to preparatory routes known in the literature. Example synthetic methods for preparing compounds of the invention are provided in the Schemes below.

Compounds of formula 4 can be synthesized using procedures as outlined in Scheme 1. Reduction of ester 1 using diisobutylaluminium hydride (DIBAL-H) can afford the corresponding aldehyde 2. Reductive amination of aldehyde 2 with aniline 3 using a suitable reducing agent such as sodium triacetoxyborohydride [Na(OAc)₃BH] in the presence of an acid such as acetic acid or trifluoroacetic acid (TFA) can afford the amine of formula 4.

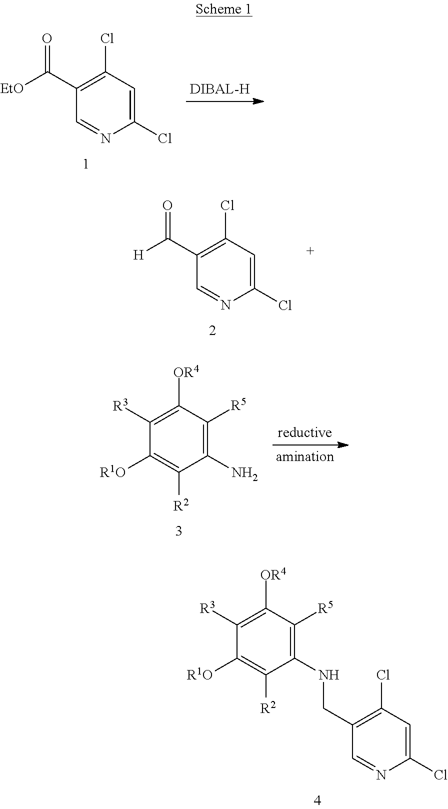

The substituted dichloropyrimidine of formula 8 can be prepared by the method described in Scheme 2. Treatment of the commercially available 5-(chloromethyl)pyrimidine-2,4(1H,3H)-dione, 5, with phosphoryl chloride (POCl₃) can afford the trichloride pyrimidine of formula 6. Compound 6 can be converted to the iodide of formula 7 using sodium iodide (NaI), tetrabutylammonium iodide (Bu₄NI), or an equivalent iodide reagent. Compound 7 can be coupled with aniline 3 in the presence of a suitable base, such as diisopropylethylamine (ⁱPr₂Net), cesium carbonate (Cs₂CO₃), or sodium hydride (NaH), to give the dichloropyrimidine of formula 8.

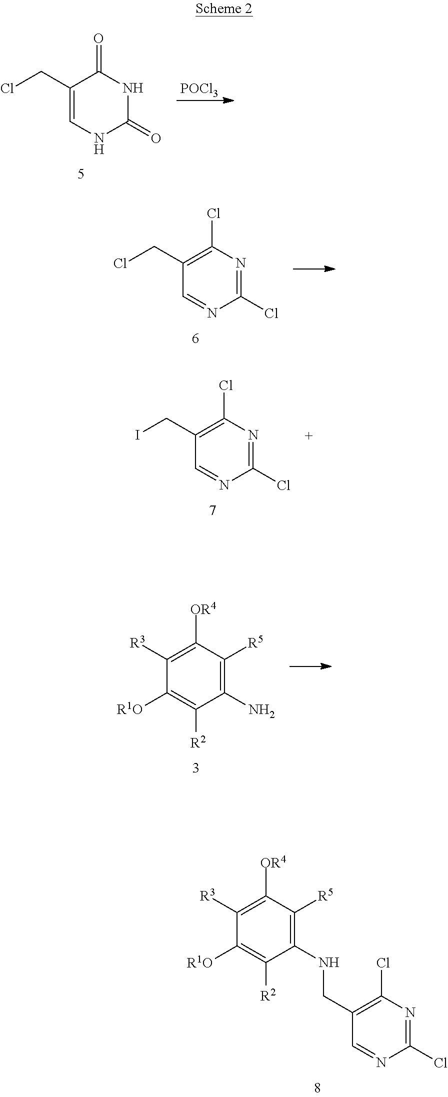

The synthesis of compound 14 is outlined in Scheme 3. Compound 9 can react with ethyl 3-chloro-3-oxopropanoate in a suitable solvent, for example tetrahydrofuran (THF), in the presence of NaH to provide the amide 10. The lactam of formula 11 can be prepared by treatment of compound 10 with a strong base (such as NaH or Cs₂CO₃) in DMF followed by an acid (such as HCl) mediated decarboxylation. The α-substituted lactam 12 can be obtained by alkylation of compound 11 with alkyl halides R¹⁰X^{hal} and/or R¹¹X^{hal} (X^{hal} is a leaving group such as Cl, Br, or I) in the presence of a base (such as NaH or Cs₂CO₃) in DMF or acetonitrile. The chloride 12 can be converted to amino-compound 13 under Buchwald-Hartwig amination conditions using suitable reagents such as Pd(OAc)₂/Xantphos/Cs₂CO₃ or Pd(OAc)₂/BrettPhos/NaOtBu, etc. The amine 13 can react with acryloyl chloride in the presence of a suitable base (such as iPr₂Net) to afford the amide 14.

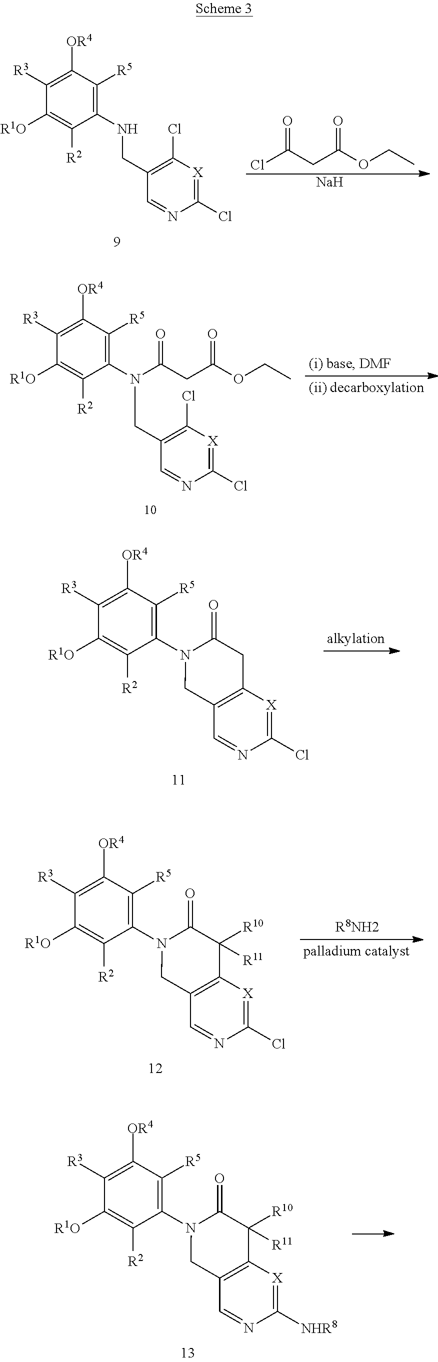

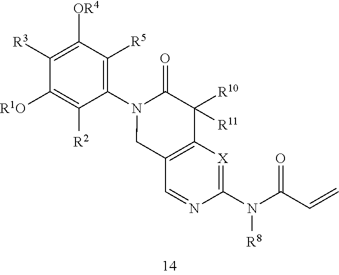

Compound 16 can be prepared by the methods described in Scheme 4. The chloride compound 12 can be converted to the corresponding amine 15 under Buchwald-Hartwig amination conditions using suitable reagents such as Pd(OAc)₂/Xantphos/Cs₂CO₃ or Pd(OAc)₂/BrettPhos/NaOtBu, etc. The amine group in compound 15 can be deprotected by removal of the protecting group, PG, under suitable conditions and can then react with acryloyl chloride, in the presence of a base, such as iPr₂NEt, to afford the amide 16. The variable L′ is L as defined herein.

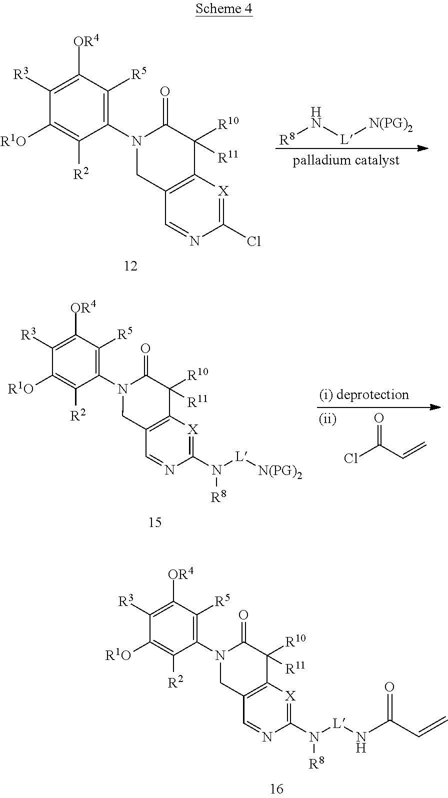

A series of aniline derivatives 21 can be prepared according to the procedures outlined in Scheme 5. Displacement of fluorine in compound 17 with benzylamine (BnNH₂) provides the aniline 18 which can be converted to bis-ether by reacting with a suitable sodium alkoxide (NaOR where R is alkyl) followed by saponification to provide acid 19. Compound 20 can be obtained by decarboxylation of benzoic acid 19, followed by hydrogenation to remove the protecting group to afford aniline 21.

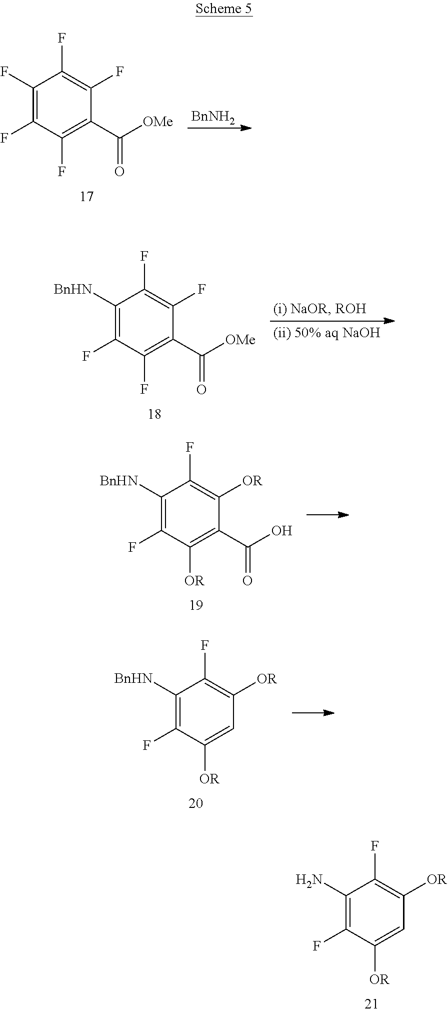

Compound 23 can be prepared by the methods described in Scheme 6. The chloride 12 can be converted to the compound 22 when treated with an amino alcohol and a strong base such as Cs₂CO₃ or NaH. The amine group in compound 22 can be deprotected by removal of the protecting group, PG, under suitable conditions and can then react with acryloyl chloride, in the presence of a base, such as iPr₂NEt, to afford the amide 23. The variable L′ is L as defined herein.

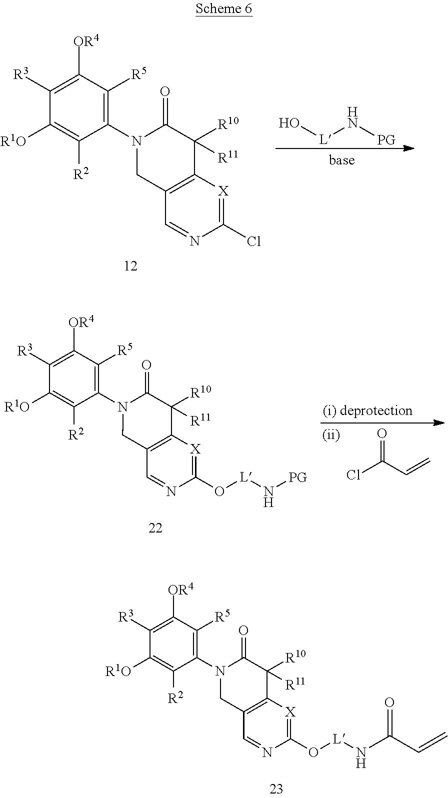

### Methods of Use

Compounds of the invention can inhibit the activity of the FGFR4 enzyme. For example, the compounds of the invention can be used to inhibit activity of an FGFR4 enzyme in a cell or in an individual or patient in need of inhibition of the enzyme by administering an inhibiting amount of a compound of the invention to the cell, individual, or patient.

In some embodiments, the compounds of the invention are selective for the enzyme FGFR4 over one or more of FGFR1, FGFR2, and/or FGFR3. In some embodiments, the compounds of the invention are selective for the enzyme FGFR4 over FGFR1, FGFR2, and FGFR3. In some embodiments, the compounds of the invention are selective for the enzyme FGFR4 over VEGFR2. In some embodiments, the selectivity is 2-fold or more, 3-fold or more, 5-fold or more, 10-fold or more, 25-fold or more, 50-fold or more, or 100-fold or more.

As FGFR4 inhibitors, the compounds of the invention are useful in the treatment of various diseases associated with abnormal expression or activity of the FGFR4 enzyme or FGFR ligands. Compounds which inhibit FGFR will be useful in providing a means of preventing the growth or inducing apoptosis in tumors, particularly by inhibiting angiogenesis. It is therefore anticipated that the compounds will prove useful in treating or preventing proliferative disorders such as cancers. In particular tumours with activating mutants of receptor tyrosine kinases or upregulation of receptor tyrosine kinases may be particularly sensitive to the inhibitors.

In certain embodiments, the FGFR4, or a mutant thereof, activity is inhibited irreversibly. In certain embodiments, FGFR4, or a mutant thereof, activity is inhibited irreversibly by covalently modifying Cys 552 of FGFR4.

In certain embodiments, the invention provides a method for treating a FGFR4-mediated disorder in a patient in need thereof, comprising the step of administering to said patient a compound according to the invention, or a pharmaceutically acceptable composition thereof.

For example, the compounds of the invention are useful in the treatment of cancer. Example cancers include bladder cancer, breast cancer, cervical cancer, colorectal cancer, cancer of the small intestine, colon cancer, rectal cancer, cancer of the anus, endometrial cancer, gastric cancer, head and neck cancer (e.g., cancers of the larynx, hypopharynx, nasopharynx, oropharynx, lips, and mouth), kidney cancer, liver cancer (e.g., hepatocellular carcinoma, cholangiocellular carcinoma), lung cancer (e.g., adenocarcinoma, small cell lung cancer and non-small cell lung carcinomas, parvicellular and non-parvicellular carcinoma, bronchial carcinoma, bronchial adenoma, pleuropulmonary blastoma), ovarian cancer, prostate cancer, testicular cancer, uterine cancer, esophageal cancer, gall bladder cancer, pancreatic cancer (e.g. exocrine pancreatic carcinoma), stomach cancer, thyroid cancer, parathyroid cancer, skin cancer (e.g., squamous cell carcinoma, Kaposi sarcoma, Merkel cell skin cancer), and brain cancer (e.g., astrocytoma, medulloblastoma, ependymoma, neuro-ectodermal tumors, pineal tumors).

Further example cancers include hematopoietic malignancies such as leukemia or lymphoma, multiple myeloma, chronic lymphocytic lymphoma, adult T cell leukemia, B-cell lymphoma, cutaneous T-cell lymphoma, acute myelogenous leukemia, Hodgkin's or non-Hodgkin's lymphoma, myeloproliferative neoplasms (e.g., polycythemia vera, essential thrombocythemia, and primary myelofibrosis), Waldenstrom's Macroglubulinemia, hairy cell lymphoma, chronic myelogenic lymphoma, acute lymphoblastic lymphoma, AIDS-related lymphomas, and Burkitt's lymphoma.

Other cancers treatable with the compounds of the invention include tumors of the eye, glioblastoma, melanoma, rhabdosarcoma, lymphosarcoma, and osteosarcoma.

The compounds of the invention can also be useful in the inhibition of tumor metastisis.

In some embodiments, the present invention provides a method for treating hepatocellular carcinoma in a patient in need thereof, comprising the step of administering to said patient a compound according to the invention, or a pharmaceutically acceptable composition thereof.

In some embodiments, the present invention provides a method for treating Rhabdomyosarcoma, esophageal cancer, breast cancer, or cancer of a head or neck, in a patient in need thereof, comprising the step of administering to said patient a compound according to the invention, or a pharmaceutically acceptable composition thereof.

In some embodiments, the present invention provides a method of treating cancer, wherein the cancer is selected from hepatocellular cancer, breast cancer, bladder cancer, colorectal cancer, melanoma, mesothelioma, lung cancer, prostate cancer, pancreatic cancer, testicular cancer, thyroid cancer, squamous cell carcinoma, glioblastoma, neuroblastoma, uterine cancer, and rhabdosarcoma.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the FGFR4 enzyme with a compound of the invention includes the administration of a compound of the present invention to an individual or patient, such as a human, having FGFR, as well as, for example, introducing a compound of the invention into a sample containing a cellular or purified preparation containing the FGFR4 enzyme.

As used herein, the term “individual” or “patient,” used interchangeably, refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) preventing the disease; for example, preventing a disease, condition or disorder in an individual who may be predisposed to the disease, condition or disorder but does not yet experience or display the pathology or symptomatology of the disease; 2) inhibiting the disease; for example, inhibiting a disease, condition or disorder in an individual who is experiencing or displaying the pathology or symptomatology of the disease, condition or disorder (i.e., arresting further development of the pathology and/or symptomatology), or 3) ameliorating the disease; for example, ameliorating a disease, condition or disorder in an individual who is experiencing or displaying the pathology or symptomatology of the disease, condition or disorder (i.e., reversing the pathology and/or symptomatology).

### Combination Therapy

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2, GM-CSF, etc.), and/or tyrosine kinase inhibitors can be used in combination with the compounds of the present invention for treatment of FGFR-associated diseases, disorders or conditions. The agents can be combined with the present compounds in a single dosage form, or the agents can be administered simultaneously or sequentially as separate dosage forms.

Suitable antiviral agents contemplated for use in combination with the compounds of the present invention can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′,3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1549. Other antiviral agents include hydroxyurea, ribavirin, IL-2, IL-12, pentafuside and Yissum Project No. 11607.

Suitable agents for use in combination with the compounds of the present invention for the treatment of cancer include chemotherapeutic agents, targeted cancer therapies, immunotherapies or radiation therapy. Compounds of this invention may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds of the present invention. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

Compounds of the present invention may be combined with or in sequence with other agents against membrane receptor kinases especially for patients who have developed primary or acquired resistance to the targeted therapy. These therapeutic agents include inhibitors or antibodies against EGFR, Her2, VEGFR, c-Met, Ret, IGFR1, or Flt-3 and against cancer-associated fusion protein kinases such as Bcr-Abl and EML4-Alk. Inhibitors against EGFR include gefitinib and erlotinib, and inhibitors against EGFR/Her2 include but are not limited to dacomitinib, afatinib, lapitinib and neratinib. Antibodies against the EGFR include but are not limited to cetuximab, panitumumab and necitumumab. Inhibitors of c-Met may be used in combination with FGFR inhibitors. These include onartumzumab, tivantnib, and INC-280. Agents against Abl (or Bcr-Abl) include imatinib, dasatinib, nilotinib, and ponatinib and those against Alk (or EML4-ALK) include crizotinib.

Angiogenesis inhibitors may be efficacious in some tumors in combination with FGFR inhibitors. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib

Activation of intracellular signaling pathways is frequent in cancer, and agents targeting components of these pathways have been combined with receptor targeting agents to enhance efficacy and reduce resistance. Examples of agents that may be combined with compounds of the present invention include inhibitors of the PI3K-AKT-mTOR pathway, inhibitors of the Raf-MAPK pathway, inhibitors of JAK-STAT pathway, and inhibitors of protein chaperones and cell cycle progression.

Agents against the PI3 kinase include but are not limited topilaralisib, idelalisib, buparlisib. Inhibitors of mTOR such as rapamycin, sirolimus, temsirolimus, and everolimus may be combined with FGFR inhibitors. Other suitable examples include but are not limited to vemurafenib and dabrafenib (Raf inhibitors) and trametinib, selumetinib and GDC-0973 (MEK inhibitors). Inhibitors of one or more JAKs (e.g., ruxolitinib, baricitinib, tofacitinib), Hsp90 (e.g., tanespimycin), cyclin dependent kinases (e.g., palbociclib), HDACs (e.g., panobinostat), PARP (e.g., olaparib), and proteasomes (e.g., bortezomib, carfilzomib) can also be combined with compounds of the present invention. In some embodiments, the JAK inhibitor is selective for JAK1 over JAK2 and JAK3.

Other suitable agents for use in combination with the compounds of the present invention include chemotherapy combinations such as platinum-based doublets used in lung cancer and other solid tumors (cisplatin or carboplatin plus gemcitabine; cisplatin or carboplatin plus docetaxel; cisplatin or carboplatin plus paclitaxel; cisplatin or carboplatin plus pemetrexed) or gemcitabine plus paclitaxel bound particles (Abraxane®).

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™), ifosfamide, melphalan, chlorambucil, pipobroman, triethylene-melamine, triethylenethiophosphoramine, busulfan, carmustine, lomustine, streptozocin, dacarbazine, and temozolomide.

Other suitable agents for use in combination with the compounds of the present invention include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Compounds according to the invention may also be combined with immunotherapy drugs, including cytokines such as interferon alpha, interleukin 2, and tumor necrosis factor (TNF) in.

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactinomycin, daunorubicin, doxorubicin, epirubicin, idarubicin, ara-C, paclitaxel (TAXOL™), mithramycin, deoxycoformycin, mitomycin-C, L-asparaginase, interferons (especially IFN-α), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4,4-1BB and PD-1, or antibodies to cytokines (IL-10, TGF-β, etc.).

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, N.J.), the disclosure of which is incorporated herein by reference as if set forth in its entirety.

### Pharmaceutical Formulations and Dosage Forms

When employed as pharmaceuticals, the compounds of the invention can be administered in the form of pharmaceutical compositions which refers to a combination of a compound of the invention, or its pharmaceutically acceptable salt, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal, or intramuscular injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

The compositions can be formulated in a unit dosage form, each dosage containing from about 5 to about 100 mg, more usually about 10 to about 30 mg, of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid pre-formulation composition containing a homogeneous mixture of a compound of the present invention. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the invention can also be formulated in combination with one or more additional active ingredients which can include any pharmaceutical agent such as anti-viral agents, vaccines, antibodies, immune enhancers, immune suppressants, anti-inflammatory agents and the like.

### Labeled Compounds and Assay Methods

Another aspect of the present invention relates to fluorescent dye, spin label, heavy metal or radio-labeled compounds of the invention that would be useful not only in imaging but also in assays, both in vitro and in vivo, for localizing and quantitating the FGFR enzyme in tissue samples, including human, and for identifying FGFR enzyme ligands by inhibition binding of a labeled compound. Accordingly, the present invention includes FGFR enzyme assays that contain such labeled compounds.

The present invention further includes isotopically-labeled compounds of the invention. An “isotopically” or “radio-labeled” compound is a compound of the invention where one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro FGFR enzyme labeling and competition assays, compounds that incorporate ³H, ¹⁴C, ⁸²Br, ¹²⁵, ¹³¹I, or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled compound” is a compound that has incorporated at least one radionuclide. In some embodiments the radionuclide is selected from the group consisting of ³H, ¹⁴C, ¹²⁵I, ³⁵S and ⁸²Br.

Synthetic methods for incorporating radio-isotopes into organic compounds are applicable to compounds of the invention and are well known in the art.

A radio-labeled compound of the invention can be used in a screening assay to identify/evaluate compounds. In general terms, a newly synthesized or identified compound (i.e., test compound) can be evaluated for its ability to reduce binding of the radio-labeled compound of the invention to the FGFR4 enzyme. Accordingly, the ability of a test compound to compete with the radio-labeled compound for binding to the FGFR4 enzyme directly correlates to its binding affinity.

### Kits

The present invention also includes pharmaceutical kits useful, for example, in the treatment or prevention of FGFR-associated diseases or disorders, obesity, diabetes and other diseases referred to herein which include one or more containers containing a pharmaceutical composition comprising a therapeutically effective amount of a compound of the invention. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples were found to be inhibitors of one or more FGFR's as described below.

### EXAMPLES

Experimental procedures for compounds of the invention are provided below. Preparatory LC-MS purifications of some of the compounds prepared were performed on Waters mass directed fractionation systems. The basic equipment setup, protocols, and control software for the operation of these systems have been described in detail in the literature. See e.g. “Two-Pump At Column Dilution Configuration for Preparative LC-MS”, K. Blom, J. Combi. Chem., 4, 295 (2002); “Optimizing Preparative LC-MS Configurations and Methods for Parallel Synthesis Purification”, K. Blom, R. Sparks, J. Doughty, G. Everlof, T. Haque, A. Combs, J. Combi. Chem., 5, 670 (2003); and “Preparative LC-MS Purification: Improved Compound Specific Method Optimization”, K. Blom, B. Glass, R. Sparks, A. Combs, J. Combi. Chem., 6, 874-883 (2004). The compounds separated were typically subjected to analytical liquid chromatography mass spectrometry (LCMS) for purity check under the following conditions: Instrument; Agilent 1100 series, LC/MSD, Column: Waters Sunfire™ C₁₈ 5 μm particle size, 2.1×5.0 mm, Buffers: mobile phase A: 0.025% TFA in water and mobile phase B: acetonitrile; gradient 2% to 80% of B in 3 minutes with flow rate 2.0 mL/minute.

Some of the compounds prepared were also separated on a preparative scale by reverse-phase high performance liquid chromatography (RP-HPLC) with MS detector or flash chromatography (silica gel) as indicated in the Examples. Typical preparative reverse-phase high performance liquid chromatography (RP-HPLC) column conditions are as follows:

pH=2 purifications: Waters Sunfire™ C₁₈ 5 μm particle size, 19×100 mm column, eluting with mobile phase A: 0.1% TFA (trifluoroacetic acid) in water and mobile phase B: acetonitrile; the flow rate was 30 mL/minute, the separating gradient was optimized for each compound using the Compound Specific Method Optimization protocol as described in the literature [see “Preparative LCMS Purification: Improved Compound Specific Method Optimization”, K. Blom, B. Glass, R. Sparks, A. Combs, J. Comb. Chem., 6, 874-883 (2004)]. Typically, the flow rate used with the 30×100 mm column was 60 mL/minute.

pH=10 purifications: Waters XBridge C₁₈ 5 μm particle size, 19×100 mm column, eluting with mobile phase A: 0.15% NH₄OH in water and mobile phase B: acetonitrile; the flow rate was 30 mL/minute, the separating gradient was optimized for each compound using the Compound Specific Method Optimization protocol as described in the literature [See “Preparative LCMS Purification: Improved Compound Specific Method Optimization”, K. Blom, B. Glass, R. Sparks, A. Combs, J. Comb. Chem., 6, 874-883 (2004)]. Typically, the flow rate used with 30×100 mm column was 60 mL/minute.

### Example 1

### N-(2-(2′-(2,6-difluoro-3,5-dimethoxyphenyl)-3′-oxo-2′,3′-dihydro-1′H-spiro[cyclopropane-1,4′-[2,7]naphthyridine]-6′-ylamino)phenyl)acrylamide

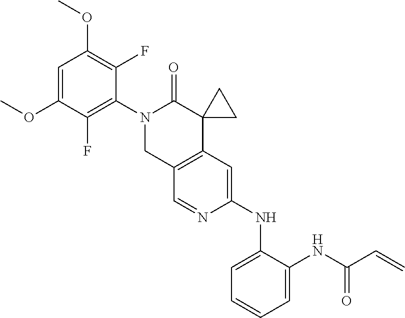 [SMILES: C=CC(=O)Nc1ccccc1Nc1cc2c(cn1)CN(c1c(F)c(OC)cc(OC)c1F)C(=O)C21CC1]

### Step 1: 4, 6-dichloronicotinaldehyde

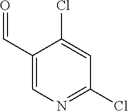

To a stirred solution of 2,4-dichloro-5-carbethoxypyridine (10.0 g, 45.4 mmol) in methylene chloride (100 mL) at −78° C. was added diisobutylaluminum hydride (1 M in DCM, 50.0 mL, 50.0 mmol) dropwise. After stirring at −78° C. for 2 hours, the reaction mixture was quenched with a saturated solution of Rochelle's salt then warmed to room temperature and stirred for 12 h. The aqueous solution was extracted with DCM (3×150 mL). The combined organic layers were dried over Na₂SO₄ and concentrated in vacuo to afford the crude aldehyde (7.51 g, 94%) which was used in the next step without further purification. LC-MS calculated for C₆H₄Cl₂NO [M+H]⁺ m/z: 176.0. found 176.0.

### Step 2: N-[(4,6-dichloropyridin-3-yl)methyl]-2,6-difluoro-3,5-dimethoxyaniline

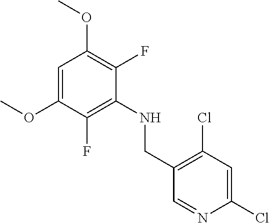 [SMILES: COc1cc(OC)c(F)c(NCc2cnc(Cl)cc2Cl)c1F]

To a stirred solution of 2,6-difluoro-3,5-dimethoxyaniline (9.03 g, 47.7 mmol), sodium triacetoxyborohydride (38.0 g, 180 mmol) in methylene chloride (60 mL)/trifluoroacetic acid (30. mL) at room temperature was added slowly a solution of 4,6-dichloronicotinaldehyde (8.00 g, 45.5 mmol) in methylene chloride (10 mL). After stirring at room temperature for 1 hour, the reaction mixture was concentrated under vacuo then saturated aqueous NaHCO₃ (200 mL) was added. The resulting mixture was extracted with DCM (3×150 mL). The organic layers were combined, dried over Na₂SO₄, and concentrated. The residue was purified on silica gel (eluting with 0 to 40% EtOAc in hexanes) to afford the desired product. LC-MS calculated for C₁₄H₁₃Cl₂F₂N₂O₂[M+H]⁺ m/z: 349.0. found 349.1.

### Step 3: ethyl 3-[[(4,6-dichloropyridin-3-yl)methyl](2,6-difluoro-3,5-dimethoxyphenyl)amino]-3-oxopropanoate

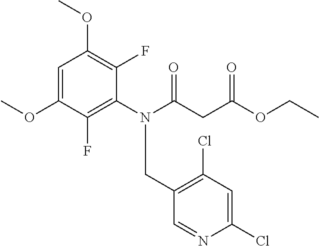 [SMILES: CCOC(=O)CC(=O)N(Cc1cnc(Cl)cc1Cl)c1c(F)c(OC)cc(OC)c1F]

To a stirred solution of N-[(4,6-dichloropyridin-3-yl)methyl]-2,6-difluoro-3,5-dimethoxyaniline (3.50 g, 10.0 mmol) in tetrahydrofuran (20 mL)) at room temperature was added NaH (60% w/w in mineral oil, 421 mg, 10.5 mmol). The resulting mixture was stirred at room temperature for 10 minutes then ethyl malonyl chloride (1.92 mL, 15.0 mmol) was added dropwise. After stirring at room temperature for 1 hour, the reaction mixture was quenched with saturated aqueous NH₄Cl, and extracted with DCM (3×100 mL). The organic layers were combined, dried over Na₂SO₄, and concentrated. The residue was purified on silica gel (eluting with 0 to 35% EtOAc in hexanes) to afford the desired product (4.20 g, 91%). LC-MS calculated for C₁₉H₁₉Cl₂F₂N₂O₅[M+H]⁺ m/z: 463.1. found 463.1.

### Step 4: 6-chloro-2-(2,6-difluoro-3,5-dimethoxyphenyl)-3-oxo-1,2,3,4-tetrahydro-2,7-naphthyridine-4-carboxylate

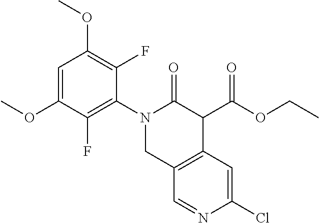 [SMILES: CCOC(=O)C1C(=O)N(c2c(F)c(OC)cc(OC)c2F)Cc2cnc(Cl)cc21]

To a stirred solution of ethyl 3-[[(4,6-dichloropyridin-3-yl)methyl](2,6-difluoro-3,5-dimethoxyphenyl)amino]-3-oxopropanoate (1.50 g, 3.24 mmol) in DMF (15 mL) at room temperature was added NaH (60% w/w in mineral oil, 337 mg, 8.42 mmol). The resulting mixture was then warmed up to 110 OC. After 5 hours, the reaction mixture was cooled to room temperature then saturated aqueous NH₄Cl (50 mL) was added. The precipitate was collected via filtration then dried under vacuo to give the crude product (0.95 g, 69%) which was used in the next step without further purification. LC-MS calculated for C₁₉H₁₈ClF₂N₂O₅ [M+H]⁺ m/z: 427.1. found 427.0.

### Step 5: 6-chloro-2-(2,6-difluoro-3,5-dimethoxyphenyl)-1,2-dihydro-2,7-naphthyridin-3(4H)-one

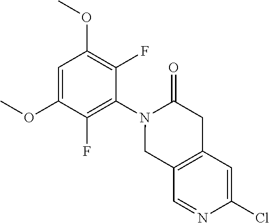 [SMILES: COc1cc(OC)c(F)c(N2Cc3cnc(Cl)cc3CC2=O)c1F]

To a stirred solution of 6-chloro-2-(2,6-difluoro-3,5-dimethoxyphenyl)-3-oxo-1,2,3,4-tetrahydro-2,7-naphthyridine-4-carboxylate(0.95 g, 2.23 mmol) in 1,4-dioxane (5 mL) at room temperature was added hydrogen chloride (4.0 M in dioxane, 2 mL, 8 mmol). The resulting mixture was warmed up to 100 OC. After 4 hours, the reaction mixture was cooled to ambient temperature, quenched with saturated aqueous NaHCO₃, and extracted with DCM (3×100 mL). The organic layers were combined, dried over Na₂SO₄, and concentrated. The residue was purified on silica gel (eluting with 0 to 30% EtOAc in DCM) to afford the desired product (0.75 g, 95%). LC-MS calculated for C₁₆H₁₄ClF₂N₂O₃ [M+H]⁺ m/z: 355.1. found 355.1.

### Step 6: 6′-chloro-2′-(2,6-difluoro-3,5-dimethoxyphenyl)-1′,2′-dihydro-3′H-spiro[cyclopropane-1,4′-[2,7]naphthyridin]-3′-one

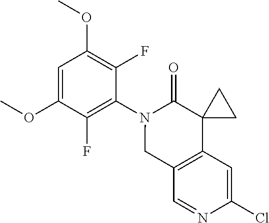 [SMILES: COc1cc(OC)c(F)c(N2Cc3cnc(Cl)cc3C3(CC3)C2=O)c1F]

To a stirred solution of 6-chloro-2-(2,6-difluoro-3,5-dimethoxyphenyl)-1,4-dihydro-2,7-naphthyridin-3(2H)-one (1.50 g, 4.23 mmol) in DMF (10 mL) at room temperature was added cesium carbonate (3.03 g, 9.30 mmol), followed by 1-bromo-2-chloro-ethane (701 μL, 8.46 mmol). After stirring at room temperature for 5 hours, the reaction mixture was quenched with saturated aqueous NH₄Cl, and extracted with DCM (3×75 mL). The organic layers were combined, dried over Na₂SO₄, and concentrated. The residue was purified on silica gel (eluting with 0 to 50% EtOAc in hexanes) to afford the desired product (1.20 g, 74%). LC-MS calculated for C₁₈H₁₆ClF₂N₂O₃ [M+H]⁺ m/z: 381.1. found 381.1.

### Step 7: 6′-[(2-aminophenyl)amino]-2′-(2,6-difluoro-3,5-dimethoxyphenyl)-1′,2′-dihydro-3′H-spiro[cyclopropane-1,4′-[2,7]naphthyridin]-3′-one

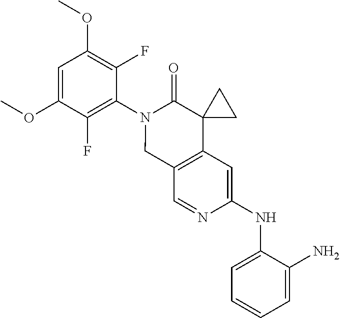 [SMILES: COc1cc(OC)c(F)c(N2Cc3cnc(Nc4ccccc4N)cc3C3(CC3)C2=O)c1F]

A stirred mixture of 1,2-benzenediamine (57 mg, 0.52 mmol), 6′-chloro-2′-(2,6-difluoro-3,5-dimethoxyphenyl)-1′,2′-dihydro-3′H-spiro[cyclopropane-1,4′-[2,7]naphthyridin]-3′-one (50.0 mg, 0.131 mmol), dicyclohexyl(2′,4′,6′-triisopropyl-3,6-dimethoxybiphenyl-2-yl)phosphine (7.1 mg, 0.013 mmol), sodium tert-butoxide (25.2 mg, 0.263 mmol), and palladium acetate (3.0 mg, 0.013 mmol) in 1,4-dioxane (3.0 mL) was heated to 110° C. under the atmosphere of N₂. After 1 hour, the reaction mixture was cooled to room temperature and quenched with saturated aq. NH₄Cl, extracted with methylene chloride. The combined organic layers were dried over Na₂SO₄ and concentrated. The residue was used directly in the next step without further purification. LC-MS calculated for C₂₄H₂₃F₂N₄O₃(M+H)⁺ m/z: 453.2. Found: 453.2.

### Step 8: N-(2-(2′-(2,6-difluoro-3,5-dimethoxyphenyl)-3′-oxo-2′,3′-dihydro-1′H-spiro[cyclopropane-1,4′-[2,7]naphthyridine]-6′-ylamino)phenyl)acrylamide

To a stirred solution of 6′-[(2-aminophenyl)amino]-2′-(2,6-difluoro-3,5-dimethoxyphenyl)-1′,2′-dihydro-3′H-spiro[cyclopropane-1,4′-[2,7]naphthyridin]-3′-one (0.020 g, 0.044 mmol) in tetrahydrofuran (2.0 mL) at 0° C. was added N,N-diisopropylethylamine (23 μL, 0.13 mmol), followed by 2-propenoyl chloride (3.5 μL, 0.044 mmol). After stirred at 0° C. for 2 minutes, the reaction mixture was quenched with saturated aq. NH₄Cl, extracted with methylene chloride. The combined organic layers were dried over Na₂SO₄ and concentrated. The residue was purified by prep-HPLC (pH=2, acetonitrile/water+TFA) to give the desired product as its TFA salt. LC-MS calculated for C₂₇H₂₅F₂N₄O₄[M+H]⁺ m/z: 507.2. found 507.2.

### Example 2

### N-(2′-(2,6-difluoro-3,5-dimethoxyphenyl)-3′-oxo-2′,3′-dihydro-1′H-spiro[cyclopropane-1,4′-[2,7]naphthyridine]-6′-yl)-N-methylacrylamide

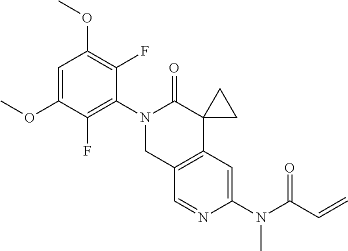 [SMILES: C=CC(=O)N(C)c1cc2c(cn1)CN(c1c(F)c(OC)cc(OC)c1F)C(=O)C21CC1]

### Step 1:2′-(2,6-difluoro-3,5-dimethoxyphenyl)-6′-(methylamino)-1′H-spiro[cyclopropane-1,4′-[2,7]naphthyridin]-3′(2′H)-one

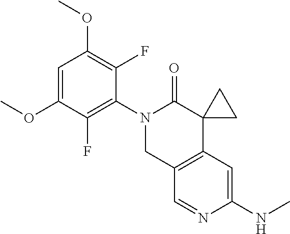

To a stirred solution of 6′-chloro-2′-(2,6-difluoro-3,5-dimethoxyphenyl)-′,2′-dihydro-3′H-spiro[cyclopropane-1,4′-[2,7]naphthyridin]-3′-one (Example 1, Step 6: 90.0 mg, 0.236 mmol) and tert-butyl methylcarbamate (89.5 mg, 0.682 mmol) in 1,4-dioxane (3 mL) at room temperature was added dicyclohexyl-(2′,4′,6′-triisopropyl-3,6-dimethoxybiphenyl-2-yl)phosphine (24.4 mg, 0.0455 mmol), sodium tert-butoxide (52.4 mg, 0.546 mmol) and palladium acetate (10.2 mg, 0.0455 mmol). The resulting mixture was purged with N₂ then heated to 90 OC. After 45 minutes, the reaction mixture was cooled to ambient temperature then concentrated under vacuo. The residue was dissolved in DCM (1 mL) then TFA (1 mL) was added. The mixture was stirred at room temperature for 1 hour then concentrated. The residue was purified by prep-HPLC (pH=2, acetonitrile/water+TFA) to give the desired product as its TFA salt. LC-MS calculated for C₁₉H₂₀F₂N₃O₃[M+H]⁺ m/z: 376.1. found 376.2. ¹H NMR (500 MHz, DMSO-d₆): δ 7.90 (s, 1H), 7.07 (t, J=10.0 Hz, 1H), 6.46 (s, 1H), 4.80 (s, 2H), 3.89 (s, 6H),), 2.90 (s, 3H) 1.79 (dd, J=10.0 Hz, 5.0 Hz, 2H), 1.56 (dd, J=10.0 Hz, 5.0 Hz, 2H) ppm.

### Step 2: N-(2′-(2,6-difluoro-3,5-dimethoxyphenyl)-3′-oxo-2′,3′-dihydro-1′H-spiro[cyclopropane-1,4′-[2,7]naphthyridine]-6′-yl)-N-methylacrylamide

To a stirred solution of 2′-(2,6-difluoro-3,5-dimethoxyphenyl)-6′-(methylamino)-1′H-spiro[cyclopropane-1,4′-[2,7]naphthyridin]-3′(2′H)-one (0.015 g, 0.040 mmol) in tetrahydrofuran (2.0 mL) at 0° C. was added N,N-diisopropylethylamine (23 μL, 0.13 mmol), followed by 2-propenoyl chloride (3.5 μL, 0.044 mmol). After stirring at 0° C. for 2 minutes, the reaction mixture was quenched with saturated aq. NH₄Cl then extracted with methylene chloride. The combined organic layers were dried over Na₂SO₄ and concentrated. The residue was purified by prep-HPLC (pH=2, acetonitrile/water+TFA) to give the desired product as its TFA salt. LC-MS calculated for C₂₂H₂₂F₂N₃O₄[M+H]⁺ m/z: 430.2. found 430.2.

### Example 3

### N-(2-(2′-(2,6-difluoro-3,5-dimethoxyphenyl)-3′-oxo-2′,3′-dihydro-1′H-spiro[cyclopropane-1,4′-[2,7]naphthyridine]-6′-ylamino)cyclohexyl)acrylamide (Racemic, cis)

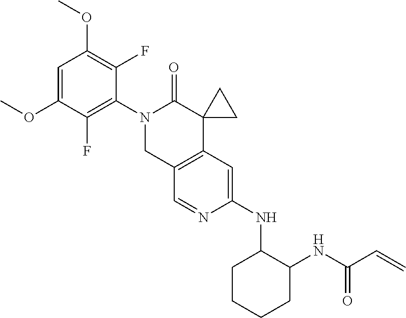 [SMILES: C=CC(=O)NC1CCCCC1Nc1cc2c(cn1)CN(c1c(F)c(OC)cc(OC)c1F)C(=O)C21CC1]

This compound was prepared using procedures analogous to those for Example 1 with racemic cis-1,2-diaminocyclohexane replacing 1,2-benzenediamine in Step 7. The product was purified by prep-HPLC (pH=2, acetonitrile/water+TFA) to give the desired compound as its TFA salt. LCMS calculated for C₂₇H₃₁F₂N₄O₄[M+H]⁺ m/z: 513.2. Found: 513.2.

### Example 4

### N-(4-(2′-(2,6-difluoro-3,5-dimethoxyphenyl)-3′-oxo-2′,3′-dihydro-1′H-spiro[cyclopropane-1,4′-[2,7]naphthyridine]-6′-ylamino)tetrahydrofuran-3-yl)acrylamide (Racemic, cis)

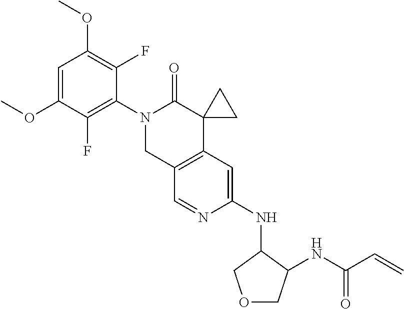 [SMILES: C=CC(=O)NC1COCC1Nc1cc2c(cn1)CN(c1c(F)c(OC)cc(OC)c1F)C(=O)C21CC1]

This compound was prepared using procedures analogous to those for Example 1 with racemic cis-tetrahydrofuran-3,4-diamine replacing 1,2-benzenediamine in Step 7. The product was purified by prep-HPLC (pH=2, acetonitrile/water+TFA) to give the desired compound as its TFA salt. LCMS calculated for C₂₅H₂₇F₂N₄O₅(M+H)+m/z: 501.2. Found: 501.2. ¹H NMR (500 MHz, DMSO-d₆): δ 8.27 (d, J=8.1 Hz, 1H), 7.92 (s, 1H), 7.16 (s, 1H), 7.06 (t, J=10.0 Hz, 1H), 6.47 (s, 1H), 6.15 (dd, J=17.1, 10.2 Hz, 1H), 5.98 (dd, J=17.1, 2.1 Hz, 1H), 5.52 (dd, J=10.2, 2.1 Hz, 1H), 4.78 (s, 2H), 4.66 (dt, J=13.9, 6.4 Hz, 1H), 4.53 (m, 1H), 4.07 (dd, J=9.1, 6.5 Hz, 1H), 4.01 (dd, J=9.0, 7.0 Hz, 1H), 3.88 (s, 6H), 3.67 (dd, J=9.5, 6.0 Hz, 1H), 3.64 (dd, J=9.5, 6.0 Hz, 1H), 1.83-1.71 (m, 2H), 1.55 (br, 2H).

### Example 5

### N-(2-(2′-(2,6-difluoro-3,5-dimethoxyphenyl)-3′-oxo-2′,3′-dihydro-1′H-spiro[cyclopropane-1,4′-[2,7]naphthyridine]-6′-ylamino)-3-methylphenyl)acrylamide

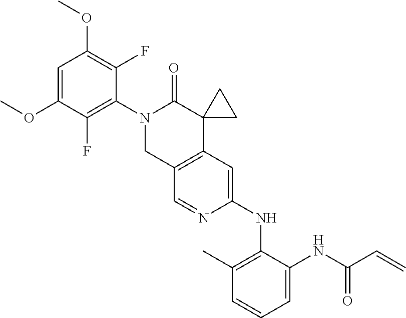 [SMILES: C=CC(=O)Nc1cccc(C)c1Nc1cc2c(cn1)CN(c1c(F)c(OC)cc(OC)c1F)C(=O)C21CC1]

### Step 1: 2′-(2,6-difluoro-3,5-dimethoxyphenyl)-6′-(2-methyl-6-nitrophenylamino)-1′H-spiro[cyclopropane-1,4′-[2,7]naphthyridin]-3′(2′H)-one

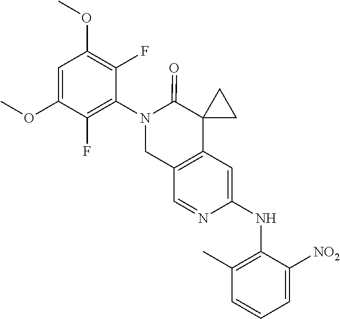 [SMILES: COc1cc(OC)c(F)c(N2Cc3cnc(Nc4c(C)cccc4[N+](=O)[O-])cc3C3(CC3)C2=O)c1F]

This compound was prepared using procedures analogous to those for Example 1, Step 7, with 2-methyl-6-nitroaniline replacing 1,2-benzenediamine. LCMS calculated for C₂₅H₂₃F₂N₄O₅ [M+H]⁺ m/z: 497.2. Found: 497.1.

### Step 2: 6′-(2-amino-6-methylphenylamino)-2′-(2,6-difluoro-3,5-dimethoxyphenyl)-1′H-spiro[cyclopropane-1,4′-[2,7]naphthyridin]-3′(2′H)-one

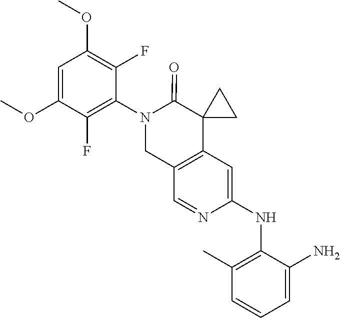 [SMILES: COc1cc(OC)c(F)c(N2Cc3cnc(Nc4c(C)cccc4N)cc3C3(CC3)C2=O)c1F]

A mixture of 2′-(2,6-difluoro-3,5-dimethoxyphenyl)-6′-[(2-methyl-6-nitrophenyl)-amino]-1′,2′-dihydro-3′H-spiro[cyclopropane-1,4′-[2,7]naphthyridin]-3′-one (50 mg, 0.1 mmol) and Pd/C (10% w/w, 11 mg, 0.010 mmol) in methanol (3.0 mL) was stirred under the atmosphere of H₂ (balloon) at room temperature. After 3 hours, the reaction mixture was filtered and the filtrate was concentrated in vacuo to afford the crude product which was used directly in the next step without further purification. LCMS calculated for C₂₅H₂₅F₂N₄O₃[M+H]⁺ m/z: 467.2. Found: 467.2.

### Step 3: N-(2-(2′-(2,6-difluoro-3,5-dimethoxyphenyl)-3′-oxo-2′,3′-dihydro-1′H-spiro[cyclopropane-1,4′-[2,7]naphthyridine]-6′-ylamino)-3-methylphenyl)acrylamide

To a stirred solution of the crude 6′-(2-amino-6-methylphenylamino)-2′-(2,6-difluoro-3,5-dimethoxyphenyl)-1′H-spiro[cyclopropane-1,4′-[2,7]naphthyridin]-3′(2′H)-one from Step 2 in tetrahydrofuran (2.0 mL) at 0° C. was added N,N-diisopropylethylamine (17.5 μL, 0.101 mmol), followed by 2-propenoyl chloride (4.1 μL, 0.050 mmol). After stirring at 0° C. for 2 minutes, the reaction mixture was quenched with saturated aq. NH₄Cl then extracted with methylene chloride. The combined organic layers were dried over Na₂SO₄ and concentrated. The residue was purified by prep-HPLC (pH=10, acetonitrile/water+NH₄OH) to give the desired product. LC-MS calculated for C₂₈H₂₇F₂N₄O₄[M+H]⁺ m/z: 521.2. found 521.2.

### Example 6

### N-(2-(7-(2,6-difluoro-3,5-dimethoxyphenyl)-5,5-dimethyl-6-oxo-5,6,7,8-tetrahydro-2,7-naphthyridin-3-ylamino)phenyl)acrylamide

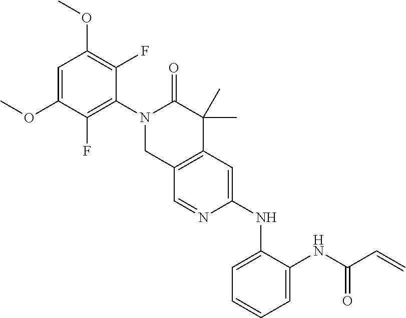 [SMILES: C=CC(=O)Nc1ccccc1Nc1cc2c(cn1)CN(c1c(F)c(OC)cc(OC)c1F)C(=O)C2(C)C]

This compound was prepared using procedures analogous to those for Example 1 with iodomethane replacing 1-bromo-2-chloro-ethane in Step 6. The product was purified by prep-HPLC (pH=2, acetonitrile/water+TFA) to give the desired compound as its TFA salt. LCMS calculated for C₂₇H₂₇F₂N₄O₄[M+H]⁺ m/z: 509.2. Found: 509.2.

### Example 7

### N-(2-(2′-(2,6-difluoro-3,5-dimethoxyphenyl)-3′-oxo-2′,3′-dihydro-1′H-spiro[cyclopentane-1,4′-[2,7]naphthyridine]-6′-ylamino)phenyl)acrylamide

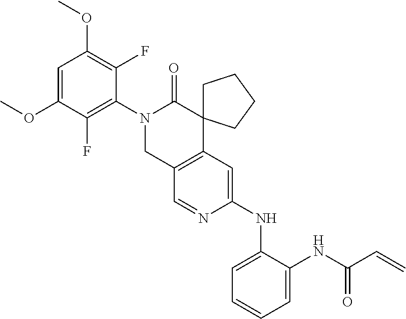 [SMILES: C=CC(=O)Nc1ccccc1Nc1cc2c(cn1)CN(c1c(F)c(OC)cc(OC)c1F)C(=O)C21CCCC1]

This compound was prepared using procedures analogous to those for Example 1, Steps 6 to 8, with 1,4-dibromobutane replacing 1-bromo-2-chloro-ethane in Step 6. The product was purified by prep-HPLC (pH=2, acetonitrile/water+TFA) to give the desired compound as its TFA salt. LCMS calculated for C₂₉H₂₉F₂N₄O₄[M+H]⁺ m/z: 535.2. Found: 535.2. ¹H NMR (500 MHz, DMSO-d₆): δ 9.72 (s, 1H), 8.89 (s, 1H), 8.01 (s, 1H), 7.70 (d, J=7.0 Hz, 1H), 7.62 (d, J=7.5 Hz, 1H), 7.23-7.13 (m, 2H), 7.05 (t, J=8.2 Hz, 1H), 6.83 (s, 1H), 6.47 (dd, J=17.0, 10.2 Hz, 1H), 6.22 (dd, J=17.0, 1.9 Hz, 1H), 5.72 (dd, J=10.2, 1.9 Hz, 1H), 4.73 (s, 2H), 3.88 (s, 6H), 2.36 (dt, J=13.1, 6.8 Hz, 2H), 1.94 (dt, J=12.3, 6.5 Hz, 2H), 1.82-1.70 (m, 4H).

### Example 8

### N-(2-(2-(2,6-difluoro-3,5-dimethoxyphenyl)-3-oxo-2,2′,3,3′,5′,6′-hexahydro-1H-spiro[[2,7]naphthyridine-4,4′-pyran]-6-ylamino)phenyl)acrylamide

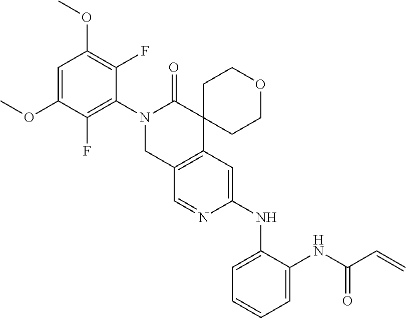 [SMILES: C=CC(=O)Nc1ccccc1Nc1cc2c(cn1)CN(c1c(F)c(OC)cc(OC)c1F)C(=O)C21CCOCC1]

This compound was prepared using procedures analogous to those for Example 1, Step 6 to 8, with 1-bromo-2-(2-bromoethoxy)ethane replacing 1-bromo-2-chloro-ethane in Step 6. The product was purified by prep-HPLC (pH=2, acetonitrile/water+TFA) to give the desired compound as its TFA salt. LCMS calculated for C₂₉H₂₉F₂N₄O₅[M+H]⁺ m/z: 551.2. Found: 551.2.

### Example 9

### N-(2-(2′-(2,6-difluoro-3,5-dimethoxyphenyl)-3′-oxo-2′,3′,4,5-tetrahydro-1′H,2H-spiro[furan-3,4′-[2,7]naphthyridine]-6′-ylamino)phenyl)acrylamide

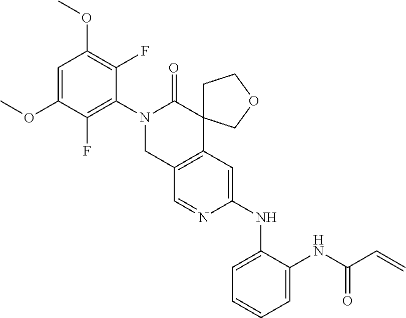 [SMILES: C=CC(=O)Nc1ccccc1Nc1cc2c(cn1)CN(c1c(F)c(OC)cc(OC)c1F)C(=O)C21CCOC1]

This compound was prepared using procedures analogous to those for Example 1, Steps 6 to 8, with 1-chloro-2-(chloromethoxy)ethane replacing 1-bromo-2-chloro-ethane in step 6. LCMS calculated for C₂₈H₂₇F₂N₄O₅[M+H]⁺ m/z: 537.2. Found: 537.1.

### Example 10

### N-(2-{[2′-(2,6-difluoro-3,5-dimethoxyphenyl)-3′-oxo-2′,3′-dihydro-1′H-spiro[cyclopropane-1,4′-[2,7]naphthyridin]-6′-yl]amino}-4-morpholin-4-ylphenyl)acrylamide

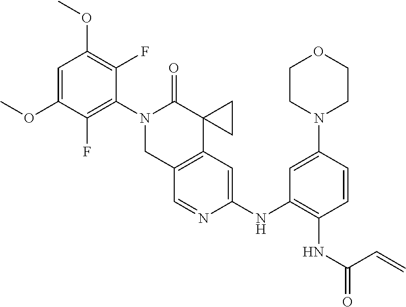 [SMILES: C=CC(=O)Nc1ccc(N2CCOCC2)cc1Nc1cc2c(cn1)CN(c1c(F)c(OC)cc(OC)c1F)C(=O)C21CC1]

### Step 1:2′-(2,6-difluoro-3,5-dimethoxyphenyl)-6′-[(5-morpholin-4-yl-2-nitrophenyl)amino]-1′,2′-dihydro-3′H-spiro[cyclopropane-1,4′-[2,7]naphthyridin]-3′-one

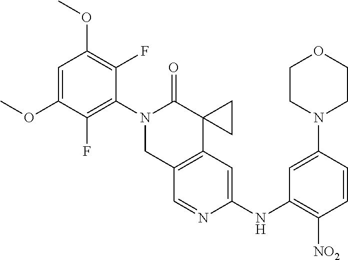 [SMILES: COc1cc(OC)c(F)c(N2Cc3cnc(Nc4cc(N5CCOCC5)ccc4[N+](=O)[O-])cc3C3(CC3)C2=O)c1F]

To a stirred mixture of 6′-chloro-2′-(2,6-difluoro-3,5-dimethoxyphenyl)-1′,2′-dihydro-3′H-spiro[cyclopropane-1,4′-[2,7]naphthyridin]-3′-one (Example 1, Step 6, 100 mg, 0.30 mmol), palladium acetate (5.9 mg, 0.026 mmol), (R)-(+)-2,2′-bis(diphenylphosphino)-1,1′-binaphthyl (16 mg, 0.026 mmol), and cesium carbonate (0.2 g, 0.5 mmol) in 1,4-dioxane (5 mL) was added 5-morpholin-4-yl-2-nitroaniline (82 mg, 0.37 mmol). The reaction mixture was stirred at 120° C. under the atmosphere of N₂ for 5 hours. After being cooled to room temperature, the reaction mixture was diluted with ethyl acetate, filtered and concentrated under reduced pressure. The residue was purified on silica gel (eluting with 0 to 0-60% EtOAc in hexanes) to afford the desired product (0.076 g). LC-MS calculated for C₂₈H₂₈F₂N₅O₆[M+H]⁺ m/z: 568.2. Found: 568.2.

### Step 2: 6′-[(2-amino-5-morpholin-4-ylphenyl)amino]-2′-(2,6-difluoro-3,5-dimethoxyphenyl)-1′,2′-dihydro-3′H-spiro[cyclopropane-1,4′-[2,7]naphthyridin]-3′-one

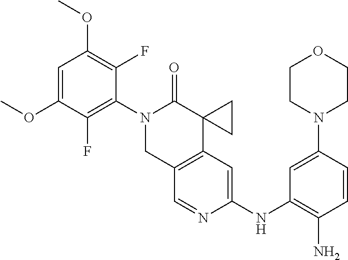 [SMILES: COc1cc(OC)c(F)c(N2Cc3cnc(Nc4cc(N5CCOCC5)ccc4N)cc3C3(CC3)C2=O)c1F]

To a stirred solution of 2′-(2,6-difluoro-3,5-dimethoxyphenyl)-6′-[(5-morpholin-4-yl-2-nitrophenyl)amino]-1′,2′-dihydro-3′H-spiro[cyclopropane-1,4′-[2,7]naphthyridin]-3′-one (76 mg, 0.13 mmol) in MeOH/4N HCl in water (1:1, 8 mL), Fe (147 mg, 2.63 mmol) was added. The resulting mixture was kept at 70° C. for 1 hour. The reaction was quenched with saturated aqueous Na₂CO₃, and extracted with ethyl acetate (3×20 mL). The combined organic layers were washed with brine, dried over MgSO₄, filtered and concentrated under reduced pressure. The residue was purified on prep-HPLC (pH=2, acetonitrile/water+TFA) to give the desired product (60 mg) as its TFA salt. LC-MS calculated for C₂₈H₃₀F₂N₅O₄[M+H]⁺ m/z: 538.2. found 538.2.

### Step 3:N-(2-{[2′-(2,6-difluoro-3,5-dim ethoxyphenyl)-3′-oxo-2′,3′-dihydro-1′H-spiro[cyclopropane-1,4′-[2,7]naphthyridin]-6′-yl]amino}-4-morpholin-4-ylphenyl)acrylamide

This compound was prepared using procedures analogous to those for Example 1, Step 8, with 6′-[(2-amino-5-morpholin-4-ylphenyl)amino]-2′-(2,6-difluoro-3,5-dimethoxyphenyl)-1′,2′-dihydro-3′H-spiro[cyclopropane-1,4′-[2,7]naphthyridin]-3′-one replacing 6′-(3-aminophenyl)-2′-(2,6-difluoro-3,5-dimethoxyphenyl)-1′H-spiro[cyclopropane-1,4′-[2,7]naphthyridin]-3′(2′H)-one. LCMS calculated for C₃₁H₃₂F₂N₅O₅[M+H]⁺ m/z: 592.2. Found: 592.3.

### Example 11

### N-(2-(2′-(2,6-difluoro-3,5-dimethoxyphenyl)-3′-oxo-2′,3′-dihydro-1′H-spiro[cyclopropane-1,4′-[2,7]naphthyridine]-6′-ylamino)-4-(4-methylpiperazin-1-yl)phenyl)acrylamide

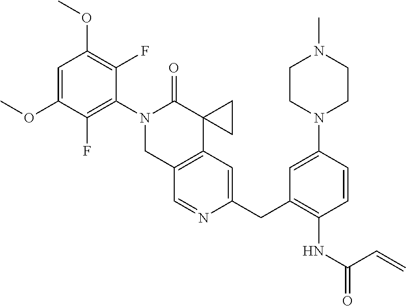 [SMILES: C=CC(=O)Nc1ccc(N2CCN(C)CC2)cc1Cc1cc2c(cn1)CN(c1c(F)c(OC)cc(OC)c1F)C(=O)C21CC1]

This compound was prepared using procedures analogous to those for Example 10, Steps 1 to 3, with 5-(4-methylpiperazin-1-yl)-2-nitroaniline replacing 5-morpholin-4-yl-2-nitroaniline in step 1. LCMS calculated for C₃₂H₃₅F₂N₆O₄[M+H]⁺ m/z: 605.3. Found: 605.3.

### Example 12

### N-((1S,2R)-2-(6′-(2,6-difluoro-3,5-dimethoxyphenyl)-7′-oxo-6′,7′-dihydro-5′H-spiro[cyclopropane-1,8′-pyrido[4,3-d]pyrimidine]-2′-yloxy)cyclopentyl)acrylamide

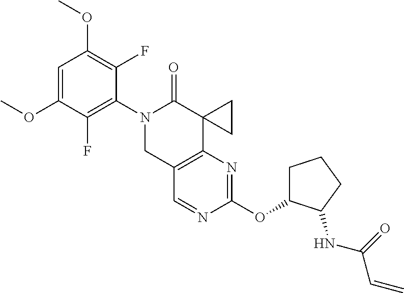

### Step 1: 2,4-dichloro-5-(chloromethyl)pyrimidine

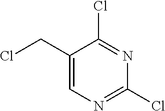

To a stirred solution of 5-(hydroxymethyl)uracil (5.0 g, 35 mmol) in phosphoryl chloride (25 mL, 270 mmol) and toluene (6.0 mL), N,N-diisopropylethylamine (26 mL, 150 mmol) was added dropwise at room temperature. The resulting solution was heated at 110 OC overnight. After being cooled to room temperature, the reaction mixture was concentrated under reduced pressure, diluted with 1N HCl (100 mL) and water (200 mL), and was extracted with DCM. The organic layers were combined, dried over Na₂SO₄, filtered and concentrated. The residue was purified on silica gel (eluting with 0-40% EtOAc in DCM) to give 6.4 g desired product. LCMS calculated for C₅H₄Cl₃N₂[M+H]⁺ m/z: 196.9. Found: 197.0.

### Step 2: 2,4-dichloro-5-(iodomethyl)pyrimidine

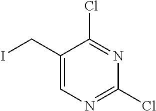

To a stirred solution of 2,4-dichloro-5-(chloromethyl)pyrimidine (1.50 g, 7.60 mmol) in acetone (10 mL), sodium iodide (1.20 g, 7.98 mmol) was added at room temperature. After 5 hours, the reaction mixture was filtered and the solid was washed with acetone. The filtrate and washed solution were combined and concentrated. The residue was purified on silica gel (eluting with 0-40% EtOAc in hexanes) to give 1.5 g desired product. LCMS calculated for C₅H₄Cl₂IN₂ [M+H]⁺ m/z: 288.9. Found: 288.8.

### Step 3: N-[(2,4-dichloropyrimidin-5-yl)methyl]-2,6-difluoro-3,5-dimethoxyaniline

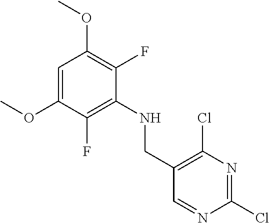 [SMILES: COc1cc(OC)c(F)c(NCc2cnc(Cl)nc2Cl)c1F]

A mixture of 2,4-dichloro-5-(iodomethyl)pyrimidine (1.50 g, 5.19 mmol), 2,6-difluoro-3,5-dimethoxyaniline (1.08 g, 5.71 mmol) in N,N-diisopropylethylamine (4 mL) was stirred at 80° C. for 2 hours. After being cooled to room temperature, the reaction mixture was concentrated under reduced pressure. The residue was purified on silica gel (eluting with 0-40% EtOAc in DCM) to give 1.70 g desired product. LCMS calculated for C₁₋₃H₁₂Cl₂F₂N₃O₂[M+H]⁺ m/z: 350.0. Found: 350.0.

### Step 4: ethyl 3-(((2,4-dichloropyrimidin-5-yl)methyl) (2,6-difluoro-3,5-dimethoxyphenyl)amino)-3-oxopropanoate

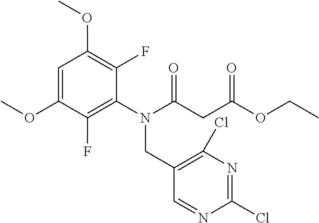 [SMILES: CCOC(=O)CC(=O)N(Cc1cnc(Cl)nc1Cl)c1c(F)c(OC)cc(OC)c1F]

This compound was prepared using procedures analogous to those for Example 1, Step 3, with N-[(2,4-dichloropyrimidin-5-yl)methyl]-2,6-difluoro-3,5-dimethoxyaniline replacing N-[(4,6-dichloropyridin-3-yl)methyl]-2,6-difluoro-3,5-dimethoxyaniline. LCMS calculated C₁₈H₁₈Cl₂F₂N₃O₅[M+H]⁺ m/z: 464.1. Found: 464.0.

### Step 5: ethyl 2-chloro-6-(2,6-difluoro-3,5-dimethoxyphenyl)-7-oxo-5,6,7,8-tetrahydropyrido[4, 3-d]pyrimidine-8-carboxylate

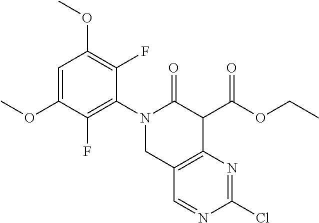 [SMILES: CCOC(=O)C1C(=O)N(c2c(F)c(OC)cc(OC)c2F)Cc2cnc(Cl)nc21]

A mixture of ethyl 3-[[(2,4-dichloropyrimidin-5-yl)methyl](2,6-difluoro-3,5-dimethoxyphenyl)amino]-3-oxopropanoate (1.2 g, 2.6 mmol) and 2-(tert-butylimino)-N,N-diethyl-1,3-dimethyl-1,3,2λ(5)-diazaphosphinan-2-amine (1.5 mL, 5.17 mmol) in methylene chloride (6 mL) was stirred at room temperature for 2 hours. The reaction mixture was concentrated under reduced pressure and the residue was purified on silica gel (eluting with 0-40% EtOAc in DCM) to give 0.88 g desired product. LCMS calculated for C₁₈H₁₇ClF₂N₃O₅ [M+H]⁺ m/z: 428.1. Found: 428.0.

### Step 6: 2-chloro-6-(2,6-difluoro-3,5-dimethoxyphenyl)-5,8-dihydropyrido[4, 3-d]pyrimidin-7(6H)-one

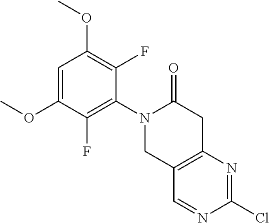 [SMILES: COc1cc(OC)c(F)c(N2Cc3cnc(Cl)nc3CC2=O)c1F]

This compound was prepared using procedures analogous to those for Example 1, Step 5, with ethyl 2-chloro-6-(2,6-difluoro-3,5-dimethoxyphenyl)-7-oxo-5,6,7,8-tetrahydropyrido[4,3-d]pyrimidine-8-carboxylate replacing 6-chloro-2-(2,6-difluoro-3,5-dimethoxyphenyl)-3-oxo-1,2,3,4-tetrahydro-2,7-naphthyridine-4-carboxylate. LCMS calculated C₁₅H₁₃ClF₂N₃O₃ [M+H]⁺ m/z: 356.1. Found: 356.1.

### Step 7: 2′-chloro-6′-(2,6-difluoro-3,5-dimethoxyphenyl)-5′,6′-dihydro-7′H-spiro[cyclopropane-1,8′-pyrido[4, 3-d]pyrimidin]-7′-one

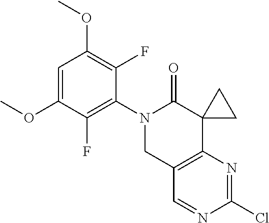 [SMILES: COc1cc(OC)c(F)c(N2Cc3cnc(Cl)nc3C3(CC3)C2=O)c1F]

This compound was prepared using procedures analogous to those for Example 1, Step 6, with 2-chloro-6-(2,6-difluoro-3,5-dimethoxyphenyl)-5,8-dihydropyrido[4,3-d]pyrimidin-7(6H)-one replacing 6-chloro-2-(2,6-difluoro-3,5-dimethoxyphenyl)-1,4-dihydro-2,7-naphthyridin-3(2H)-one. LCMS calculated C₁₇H₁₅ClF₂N₃O₃ [M+H]⁺ m/z: 382.1. Found: 382.0.

### Step 8: tert-butyl[(1S,2R)-2-hydroxycyclopentyl]carbamate

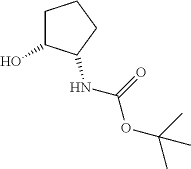

To a stirred solution of (1R,2S)-2-aminocyclopentanol hydrochloride (50.0 mg, 0.363 mmol) in methylene chloride (3.0 mL), N,N-diisopropylethylamine (0.19 mL, 1.09 mmol) and di-tert-butyl carbonate (63.3 mg, 0.363 mmol) were added sequentially at room temperature. After 2 hours, the volatiles were removed and the residue (70 mg) was used directly in the next step without further purification.

### Step 9: 2′-{[(1R,2S)-2-aminocyclopentyl]oxy}-6′-(2,6-difluoro-3,5-dimethoxyphenyl)-5′,6′-dihydro-7′H-spiro[cyclopropane-1,8′-pyrido[4, 3-d]pyrimidin]-7′-one

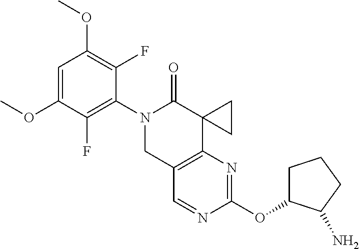

To a stirred solution of crude tert-butyl[(1S,2R)-2-hydroxycyclopentyl]carbamate (70 mg) and 2′-chloro-6′-(2,6-difluoro-3,5-dimethoxyphenyl)-5′,6′-dihydro-7′H-spiro[cyclopropane-1,8′-pyrido[4,3-d]pyrimidin]-7′-one (139 mg, 0.364 mmol) in acetonitrile (5.0 mL), sodium hydride (0.0320 g, 0.799 mmol) was added at room temperature. The resulting mixture was warmed up to 80° C. After 2 hours, the reaction was quenched with saturated aqueous NH₄Cl, extracted with methylene chloride. The combined organic layers were dried over Na₂SO₄, filtered, and concentrated to dryness under reduced pressure. The crude product was purified on prep-HPLC (pH=2, acetonitrile/water+TFA) to give the desired product (20 mg) as its TFA salt. LC-MS calculated for C₂₂H₂₅F₂N₄O₄[M+H]⁺ m/z: 447.2. found 447.2.

### Step 10: N-((1S,2R)-2-(6′-(2,6-difluoro-3,5-dimethoxyphenyl)-7′-oxo-6′,7′-dihydro-5′H-spiro[cyclopropane-1,8′-pyrido[4, 3-d]pyrimidine]-2′-yloxy)cyclopentyl)acrylamide

This compound was prepared using procedures analogous to those for Example 1, Step 8, with 2′-{[(1R,2S)-2-aminocyclopentyl]oxy}-6′-(2,6-difluoro-3,5-dimethoxyphenyl)-5′,6′-dihydro-7′H-spiro[cyclopropane-1,8′-pyrido[4,3-d]pyrimidin]-7′-one replacing 6′-(3-aminophenyl)-2′-(2,6-difluoro-3,5-dimethoxyphenyl)-1′H-spiro[cyclopropane-1,4′-[2,7]naphthyridin]-3′(2′H)-one. LCMS calculated for C₂₇H₂₇F₂N₄O₅[M+H]⁺ m/z: 501.2. Found: 501.2.

### Example A

### FGFR Enzymatic Assay

The inhibitor potency of the exemplified compounds was measured in an enzyme assay that measures peptide phosphorylation using FRET measurements to detect product formation. Inhibitors were serially diluted in DMSO and a volume of 0.5 μL was transferred to the wells of a 384-well plate. For FGFR3, a 10 μL volume of FGFR3 enzyme (Millipore) diluted in assay buffer (50 mM HEPES, 10 mM MgCl₂, 1 mM EGTA, 0.01% Tween-20, 5 mM DTT, pH 7.5) was added to the plate and pre-incubated for a time between 5-10 minutes and up to 4 hours. Appropriate controls (enzyme blank and enzyme with no inhibitor) were included on the plate. The assay was initiated by the addition of a 10 L solution containing biotinylated EQEDEPEGDYFEWLE peptide substrate (SEQ ID NO: 1) and ATP (final concentrations of 500 nM and 140 μM respectively) in assay buffer to the wells. The plate was incubated at 25° C. for 1 hr. The reactions were ended with the addition of 10 μL/well of quench solution (50 mM Tris, 150 mM NaCl, 0.5 mg/mL BSA, pH 7.8; 30 mM EDTA with Perkin Elmer Lance Reagents at 3.75 nM Eu-antibody PY20 and 180 nM APC-Streptavidin). The plate was allowed to equilibrate for ˜1 hr before scanning the wells on a PheraStar plate reader (BMG Labtech).

FGFR1, FGFR2, and FGFR4 are measured under equivalent conditions with the following changes in enzyme and ATP concentrations: FGFR1, 0.02 nM and 210 uM respectively, FGFR2, 0.01 nM and 100 uM, respectively, and FGFR4, 0.04 nM and 600 uM respectively. The enzymes were purchased from Millipore or Invitrogen.

GraphPad prism3 was used to analyze the data. The IC₅₀ values were derived by fitting the data to the equation for a sigmoidal dose-response with a variable slope. Y=Bottom+(Top−Bottom)/(1+10̂((Log IC₅₀−X)*HillSlope)) where X is the logarithm of concentration and Y is the response. Compounds having an IC₅₀ of 1 μM or less are considered active.

The compounds of the invention were found to be selective inhibitors of FGFR4 according to the FGFR Enzymatic Assay. Tables 1 and 2 provide IC₅₀ data for compounds of the invention assayed in the FGFR Enzymatic Assay after dilution in assay buffer, added to the plate and pre-incubated for 4 hours. The symbol: “+” indicates an IC₅₀ less than 10 nM; “++” indicates an IC₅₀ greater than or equal to 10 nM but less than 30 nM; “+++” indicates an IC₅₀ greater than or equal to 30 nM but less than 200 nM; and “++++” indicates an IC₅₀ greater than or equal to 200 nM.

TABLE 1

| | FGFR1 | FGFR2 | FGFR3 | FGFR4 |
|---|---|---|---|---|
| Example No. | IC50 (nM) | IC50 (nM) | IC50 (nM) | IC50 (nM) |
| 1 | +++ | +++ | +++ | + |
| 2 | +++ | +++ | +++ | + |
| 3 | ++++ | ++++ | ++++ | + |
| 4 | +++ | ++++ | ++++ | + |
| 5 | ++++ | ++++ | ++++ | + |
| 6 | ++++ | ++++ | ++++ | + |
| 7 | +++ | +++ | ++++ | + |
| 8 | ++++ | ++++ | ++++ | + |
| 9 | ++++ | ++++ | ++++ | + |
| 10 | +++ | ++++ | +++ | + |
| 11 | +++ | +++ | ++++ | + |
| 12 | ++++ | ++++ | ++++ | ++ |

TABLE 2

| | FGFR1/ | FGFR2/ | FGFR3/ |
|---|---|---|---|
| Example No. | FGFR4 | FGFR4 | FGFR4 |
| 1 | >100 | >100 | >100 |
| 2 | >10 | >5 | >10 |
| 3 | >25 | >25 | >100 |
| 4 | >100 | >100 | >100 |
| 5 | >100 | >100 | >100 |
| 6 | >100 | >100 | >100 |
| 7 | >100 | >100 | >100 |
| 8 | >100 | >100 | >100 |
| 9 | >100 | >100 | >100 |
| 10 | >100 | >100 | >100 |
| 11 | >25 | >25 | >100 |
| 12 | >100 | >100 | >100 |

Tables 3 and 4 provide IC₅₀ data for compounds of the invention assayed in the FGFR Enzymatic Assay after dilution in assay buffer, added to the plate and pre-incubated for 5 to 10 minutes. The symbol: “+” indicates an IC₅₀ less than 10 nM; “++” indicates an IC₅₀ greater than or equal to 10 nM but less than 30 nM; “+++” indicates an IC₅₀ greater than or equal to 30 nM but less than 200 nM; and “++++” indicates an IC₅₀ greater than or equal to 200 nM.

TABLE 3

| | FGFR1 | FGFR2 | FGFR3 | FGFR4 |
|---|---|---|---|---|
| Example No. | IC50 (nM) | IC50 (nM) | IC50 (nM) | IC50 (nM) |
| 1 | +++ | +++ | +++ | + |
| 2 | +++ | +++ | ++++ | ++ |
| 3 | ++++ | ++++ | ++++ | +++ |
| 4 | ++++ | ++++ | ++++ | + |
| 5 | ++++ | ++++ | ++++ | + |
| 6 | ++++ | ++++ | ++++ | ++ |
| 7 | +++ | ++++ | ++++ | + |
| 8 | ++++ | ++++ | ++++ | ++++ |
| 9 | ++++ | ++++ | ++++ | ++ |
| 10 | ++++ | ++++ | +++ | + |
| 11 | ++++ | ++++ | ++++ | + |
| 12 | ++++ | ++++ | ++++ | +++ |

TABLE 4

| | FGFR1/ | FGFR2/ | FGFR3/ |
|---|---|---|---|
| Example No. | FGFR4 | FGFR4 | FGFR4 |
| 1 | >10 | >10 | >10 |
| 2 | >5 | >2 | >5 |
| 3 | >5 | >5 | >10 |
| 4 | >10 | >10 | >10 |
| 5 | >10 | >10 | >10 |
| 6 | >10 | >10 | >10 |
| 7 | >10 | >10 | >10 |
| 8 | >10 | >10 | >10 |
| 9 | >10 | >10 | >10 |
| 10 | >10 | >10 | >10 |
| 11 | >10 | >10 | >10 |
| 12 | >10 | >10 | >10 |

### Example B

### FGFR4 Cellular and In Vivo Assays

The FGFR4 inhibitory activity of the example compounds in cells, tissues, and/or animals can be demonstrated according to one or more assays or models described in the art such as, for example, in French et al. “Targeting FGFR4 Inihibits Hepatocellular Carcinoma in Preclinical Mouse Models,” PLoS ONE, May 2012, Vol. 7, Issue 5, e36713, which is incorporated herein by reference in its entirety.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula (I′): 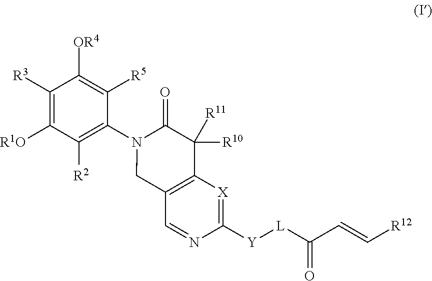
or a pharmaceutically acceptable salt thereof, wherein:
X is N or CR⁶;
Y is O or NR⁸;
R¹ is C₁₋₃ alkyl or C₁₋₃ haloalkyl;
R² is H, halo, C₁₋₃ alkyl, C₁₋₃ haloalkyl, CN, or C₁₋₃ alkoxy;
R³ is H, halo, C₁₋₃ alkyl, C₁₋₃ haloalkyl, CN, or C₁₋₃ alkoxy;
R⁴ is C₁₋₃ alkyl or C₁₋₃ haloalkyl;
R⁵ is H, halo, C₁₋₃ alkyl, C₁₋₃ haloalkyl, CN, or C₁₋₃ alkoxy;
R⁶ is H, halo, CN, OR^{a4}, SR^{a4}, C(O)NR^{c4}R^{d4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, C₁₋₆ alkyl, C₂₋₆ alkenyl, C₂₋₆ alkynyl, C₁₋₆ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, phenyl, C₃₋₆ cycloalkyl, 5-6 membered heteroaryl, and 4-7 membered heterocycloalkyl groups of R⁶ are each optionally substituted with 1, 2, or 3 substituents independently selected from R^{10A};
L is a bond or *—CR^{7A}R^{7B} [symbol] CR^{7C}R^{7D}—NR^{7E}—, wherein the symbol * indicates the point of attachment to Y in Formula (I′); wherein the symbol [symbol] represents a single or double bond; and wherein R^{7B} and R^{7D} are absent when [symbol] is a double bond;
R^{7A} is selected from H, C₁₋₄ alkyl, C₂₋₄ alkenyl, C₂₋₄ alkynyl, phenyl, C₃₋₇ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₇ cycloalkyl, 5-6 membered heteroaryl, and 4-7 membered heterocycloalkyl groups of R^{7A} are each optionally substituted with 1, 2, or 3 substituents independently selected from R¹⁷;
R^{7B} is H or C₁₋₄ alkyl optionally substituted with 1, 2, or 3 substituents independently selected from R¹⁷;
or R^{7A} and R^{7B} together with the carbon atom to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl and 4-7 membered heterocycloalkyl groups are each optionally substituted with 1, 2, or 3 substituents independently selected from R¹⁷;
R^{7C} is selected from H, C₁₋₄ alkyl, C₂₋₄ alkenyl, C₂₋₄ alkynyl, phenyl, C₃₋₇ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₇ cycloalkyl, 5-6 membered heteroaryl, and 4-7 membered heterocycloalkyl group of R^{7C} are each optionally substituted with 1, 2, or 3 substituents independently selected from R¹⁷;
R^{7D} is H or C₁₋₄ alkyl optionally substituted with 1, 2, or 3 substituents independently selected from R¹⁷;
or R^{7C} and R^{7D} together with the carbon atom to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl and 4-7 membered heterocycloalkyl groups are each optionally substituted with 1, 2, or 3 substituents independently selected from R¹⁷;
R^{7E} is selected from H, C₁₋₄ alkyl, C₂₋₄ alkenyl, and C₂₋₄ alkynyl;
alternatively, when [symbol] is a double bond, R^{7A} and R^{7C} together with the carbon atoms to which they are attached form a phenyl group or a 5-6 membered heteroaryl moiety comprising carbon and 1, 2, or 3 heteroatoms independently selected from N, O and S, wherein said phenyl and 5-6 membered heteroaryl groups are each optionally substituted with 1, 2, or 3 substituents independently selected from R¹⁷;
alternatively, when [symbol] is a single bond, R^{7A} and R^{7C} together with the carbon atoms to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl group and 4-7 membered heterocycloalkyl group are each optionally substituted with 1, 2, or 3 substituents independently selected from R¹⁷;
alternatively, R^{7A} and R^{7E} together with the carbon and nitrogen atoms to which they are attached form a 5-6 membered heteroaryl moiety comprising carbon and 1, 2, or 3 nitrogen atoms, or a 4-7 membered heterocycloalkyl moiety comprising carbon and 1 or 2 nitrogen atoms; wherein said 5-6 membered heteroaryl and 4-7 membered heterocycloalkyl group are each optionally substituted with 1, 2, or 3 substituents independently selected from R¹⁷;
alternatively, R^{7C} and R^{7E} together with the carbon and nitrogen atoms to which they are attached form a 5-6 membered heteroaryl moiety comprising carbon and 1, 2, or 3 nitrogen atoms, or a 4-7 membered heterocycloalkyl moiety comprising carbon and 1 or 2 nitrogen atoms; wherein said 5-6 membered heteroaryl and 4-7 membered heterocycloalkyl group are each optionally substituted with 1, 2, or 3 substituents independently selected from R¹⁷;
R⁸ is H or C₁₋₄ alkyl which is optionally substituted by halo, CN, OR^{a9}, C(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, S(O)₂NR^{c9}R^{d9}, phenyl, C₃₋₇ cycloalkyl, a 5-6 membered heteroaryl moiety comprising carbon and 1, 2, or 3 heteroatoms independently selected from N, O and S, or a 4-7 membered heterocycloalkyl moiety comprising carbon and 1, 2, or 3 heteroatoms independently selected from N, O and S; wherein said phenyl, C₃₋₇ cycloalkyl, 5-6 membered heteroaryl, and 4-7 membered heterocycloalkyl groups of R⁸ are each optionally substituted with 1 or 2 R¹⁹;
R¹⁰ is selected from C₁₋₆ alkyl, C₂₋₆ alkenyl, C₂₋₆ alkynyl, C₁₋₆ haloalkyl, C₆₋₁₀ aryl, C₃₋₁₀ cycloalkyl, a 5-10 membered heteroaryl moiety comprising carbon and 1, 2, or 3 heteroatoms independently selected from N, O and S, and a 4-10 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl groups of R¹⁰ are each optionally substituted with 1, 2, 3, or 4 R^{10A};
R^{10A}, at each occurrence, is independently selected from halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4} S(O)₂NR^{c4}R^{d4}, C₁₋₆ alkyl, C₂₋₆ alkenyl, C₂₋₆ alkynyl, C₁₋₆ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, phenyl, C₃₋₆ cycloalkyl, 5-6 membered heteroaryl, and 4-7 membered heterocycloalkyl group of R^{10A} are each optionally substituted with 1, 2, or 3 substituents independently selected from R¹⁹;
R^{a4}, R^{b4}, R^{c4}, and R^{d4}, at each occurrence, are independently selected from H, C₁₋₄ alkyl, C₂₋₄ alkenyl, C₂₋₄ alkynyl, C₁₋₄ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₆ cycloalkyl, 5-6 membered heteroaryl, and 4-7 membered heterocycloalkyl group of R^{a4}, R^{b4}, R^{c4}, and R^{d4} are each optionally substituted with 1, 2, or 3 substituents independently selected from R¹⁹;
alternatively, R^{c4} and R^{d4} together with the nitrogen atom to which they are attached form a 4-, 5-, 6-, or 7-membered heterocycloalkyl group which is optionally substituted with 1, 2 or 3 substituents independently selected from R¹⁹;
R^{e4} is H or C₁₋₄ alkyl;
R¹¹ is selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl, and C₂₋₆ alkynyl are each optionally substituted with 1, 2 or 3 substituents independently selected from R¹⁹;
alternatively, R¹⁰ and R¹¹ together with the carbon atom to which they are attached form a 3-, 4-, 5-, 6-, or 7-membered cycloalkyl group or a 4-, 5-, 6-, 7-, 8-, 9-, or 10-membered heterocycloalkyl group; wherein said 3-, 4-, 5-, 6-, or 7-membered cycloalkyl group and 4-, 5-, 6-, 7-, 8-, 9-, or 10-membered heterocycloalkyl group are each optionally substituted with 1, 2, 3 or 4 R^{10A};
R¹² is H or C₁₋₄ alkyl which is optionally substituted by R¹⁷;
R¹⁷, at each occurrence, is independently selected from halo, CN, NO₂, OR^{a7}, SR^{a7}, C(O)R^{b7}, C(O)NR^{c7}R^{d7}, C(O)OR^{a7}, OC(O)R^{b7}, OC(O)NR^{c7}R^{d7}, C(═NR^{e7})NR^{c7}R^{d7}, NR^{c7}C(═NR^{e7})NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)R^{b7}, NR^{c7}S(O)₂R^{b7}, NR^{c7}S(O)₂NR^{c7}R^{d7}, S(O)R^{b7}, S(O)NR^{c7}R^{d7}, S(O)₂R^{b7}, S(O)₂NR^{c7}R^{d7}, C₁₋₆ alkyl, C₂₋₆ alkenyl, C₂₋₆ alkynyl, C₁₋₆ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, phenyl, C₃₋₆ cycloalkyl, 5-6 membered heteroaryl, and 4-7 membered heterocycloalkyl groups of R¹⁷ are each optionally substituted with 1, 2, or 3 substituents independently selected from R¹⁹;
R^{a7}, R^{b7}, R^{c7}, and R^{d7}, at each occurrence, are independently selected from H, C₁₋₄ alkyl, C₂₋₄ alkenyl, C₂₋₄ alkynyl, C₁₋₄ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₆ cycloalkyl, 5-6 membered heteroaryl, and 4-7 membered heterocycloalkyl groups of R^{a7}, R^{b7}, R^{c7}, and R^{d7} are each optionally substituted with 1, 2, or 3 substituents independently selected from R¹⁹;
alternatively, R^{c7} and R^{d7} together with the nitrogen atom to which they are attached form a 4-, 5-, 6-, or 7-membered heterocycloalkyl group which is optionally substituted with 1, 2 or 3 substituents independently selected from R¹⁹;
R^{e7} is H or C₁₋₄ alkyl;
R¹⁹, at each occurrence, is independently selected from halo, CN, NO₂, OR^{a9}, SR^{a9}, C(O)R^{b9}, C(O)NR^{c9}R^{d9}, C(O)OR^{a9}, OC(O)R^{b9}, OC(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, S(O)₂NR^{c9}R^{d9}, C₁₋₄ alkyl, C₂₋₄ alkenyl, C₂₋₄ alkynyl, and C₁₋₄ haloalkyl;
R^{a9}, R^{c9}, and R^{d9}, at each occurrence, are independently selected from H and C₁₋₄ alkyl; and
R^{b9} is C₁₋₄ alkyl.

2. The compound of claim 1, having Formula (I): 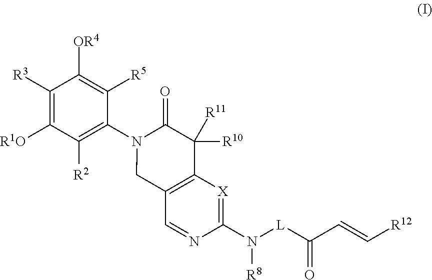
or a pharmaceutically acceptable salt thereof, wherein:
X is N or CR⁶;
R¹ is C₁₋₃ alkyl or C₁₋₃ haloalkyl;
R² is H, halo, C₁₋₃ alkyl, C₁₋₃ haloalkyl, CN, or C₁₋₃ alkoxy;
R³ is H, halo, C₁₋₃ alkyl, C₁₋₃ haloalkyl, CN, or C₁₋₃ alkoxy;
R⁴ is C₁₋₃ alkyl or C₁₋₃ haloalkyl;
R⁵ is H, halo, C₁₋₃ alkyl, C₁₋₃ haloalkyl, CN, or C₁₋₃ alkoxy;
R⁶ is H, halo, CN, OR^{a4}, SR^{a4}, C(O)NR^{c4}R^{d4}, OC(O)NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, C₁₋₆ alkyl, C₂₋₆ alkenyl, C₂₋₆ alkynyl, C₁₋₆ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, phenyl, C₃₋₆ cycloalkyl, 5-6 membered heteroaryl, and 4-7 membered heterocycloalkyl groups of R⁶ are each optionally substituted with 1, 2, or 3 substituents independently selected from R^{10A};
L is a bond or *—CR^{7A}R^{7B} [symbol] CR^{7C}R^{7D}—NR^{7E}—, wherein the symbol * indicates the point of attachment to NR⁸ in Formula (I); wherein the symbol [symbol] represents a single or double bond; and wherein R^{7B} and R^{7D} are absent when [symbol] is a double bond;
R^{7A} is selected from H, C₁₋₄ alkyl, C₂₋₄ alkenyl, C₂₋₄ alkynyl, phenyl, C₃₋₇ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₇ cycloalkyl, 5-6 membered heteroaryl, and 4-7 membered heterocycloalkyl groups of R^{7A} are each optionally substituted with 1, 2, or 3 substituents independently selected from R¹⁷;
R^{7B} is H or C₁₋₄ alkyl optionally substituted with 1, 2, or 3 substituents independently selected from R¹⁷;
or R^{7A} and R^{7B} together with the carbon atom to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl and 4-7 membered heterocycloalkyl groups are each optionally substituted with 1, 2, or 3 substituents independently selected from R¹⁷;
R^{7C} is selected from H, C₁₋₄ alkyl, C₂₋₄ alkenyl, C₂₋₄ alkynyl, phenyl, C₃₋₇ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₇ cycloalkyl, 5-6 membered heteroaryl, and 4-7 membered heterocycloalkyl group of R^{7C} are each optionally substituted with 1, 2, or 3 substituents independently selected from R¹⁷;
R^{7D} is H or C₁₋₄ alkyl optionally substituted with 1, 2, or 3 substituents independently selected from R¹⁷;
or R^{7C} and R^{7D} together with the carbon atom to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl and 4-7 membered heterocycloalkyl groups are each optionally substituted with 1, 2, or 3 substituents independently selected from R¹⁷;
R^{7E} is selected from H, C₁₋₄ alkyl, C₂₋₄ alkenyl, and C₂₋₄ alkynyl;
alternatively, when [symbol] is a double bond, R^{7A} and R^{7C} together with the carbon atoms to which they are attached form a phenyl group or a 5-6 membered heteroaryl moiety comprising carbon and 1, 2, or 3 heteroatoms independently selected from N, O and S, wherein said phenyl and 5-6 membered heteroaryl groups are each optionally substituted with 1, 2, or 3 substituents independently selected from R¹⁷;
alternatively, when [symbol] is a single bond, R^{7A} and R^{7C} together with the carbon atoms to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl group and 4-7 membered heterocycloalkyl group are each optionally substituted with 1, 2, or 3 substituents independently selected from R¹⁷;
alternatively, R^{7A} and R^{7E} together with the carbon and nitrogen atoms to which they are attached form a 5-6 membered heteroaryl moiety comprising carbon and 1, 2, or 3 nitrogen atoms, or a 4-7 membered heterocycloalkyl moiety comprising carbon and 1 or 2 nitrogen atoms; wherein said 5-6 membered heteroaryl and 4-7 membered heterocycloalkyl group are each optionally substituted with 1, 2, or 3 substituents independently selected from R¹⁷;
alternatively, R^{7C} and R^{7E} together with the carbon and nitrogen atoms to which they are attached form a 5-6 membered heteroaryl moiety comprising carbon and 1, 2, or 3 nitrogen atoms, or a 4-7 membered heterocycloalkyl moiety comprising carbon and 1 or 2 nitrogen atoms; wherein said 5-6 membered heteroaryl and 4-7 membered heterocycloalkyl group are each optionally substituted with 1, 2, or 3 substituents independently selected from R¹⁷;
R⁸ is H or C₁₋₄ alkyl which is optionally substituted by halo, CN, OR^{a9}, C(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, S(O)₂NR^{c9}R^{d9}, phenyl, C₃₋₇ cycloalkyl, a 5-6 membered heteroaryl moiety comprising carbon and 1, 2, or 3 heteroatoms independently selected from N, O and S, or a 4-7 membered heterocycloalkyl moiety comprising carbon and 1, 2, or 3 heteroatoms independently selected from N, O and S; wherein said phenyl, C₃₋₇ cycloalkyl, 5-6 membered heteroaryl, and 4-7 membered heterocycloalkyl groups of R⁸ are each optionally substituted with 1 or 2 R¹⁹;
R¹⁰ is selected from C₁₋₆ alkyl, C₂₋₆ alkenyl, C₂₋₆ alkynyl, C₁₋₆ haloalkyl, C₆₋₁₀ aryl, C₃₋₁₀ cycloalkyl, a 5-10 membered heteroaryl moiety comprising carbon and 1, 2, or 3 heteroatoms independently selected from N, O and S, and a 4-10 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, C₆-10 aryl, C₃₋₁₀ cycloalkyl, 5-10 membered heteroaryl, and 4-10 membered heterocycloalkyl groups of R¹⁰ are each optionally substituted with 1, 2, 3, or 4 R^{10A};
R^{10A}, at each occurrence, is independently selected from halo, CN, NO₂, OR^{a4}, SR^{a4}, C(O)R^{b4}, C(O)NR^{c4}R^{d4}, C(O)OR^{a4}, OC(O)R^{b4}, OC(O)NR^{c4}R^{d4}, C(═NR^{e4})NR^{c4}R^{d4}, NR^{c4}C(═NR^{e4})NR^{c4}R^{d4}, NR^{c4}R^{d4}, NR^{c4}C(O)R^{b4}, NR^{c4}C(O)OR^{a4}, NR^{c4}C(O)NR^{c4}R^{d4}, NR^{c4}S(O)R^{b4}, NR^{c4}S(O)₂R^{b4}, NR^{c4}S(O)₂NR^{c4}R^{d4}, S(O)R^{b4}, S(O)NR^{c4}R^{d4}, S(O)₂R^{b4}, S(O)₂NR^{c4}R^{d4}, C₁₋₆ alkyl, C₂₋₆ alkenyl, C₂₋₆ alkynyl, C₁₋₆ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, phenyl, C₃₋₆ cycloalkyl, 5-6 membered heteroaryl, and 4-7 membered heterocycloalkyl group of R^{10A} are each optionally substituted with 1, 2, or 3 substituents independently selected from R¹⁹;
R^{a4}, R^{b4}, R^{c4}, and R^{d4}, at each occurrence, are independently selected from H, C₁₋₄ alkyl, C₂₋₄ alkenyl, C₂₋₄ alkynyl, C₁₋₄ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₆ cycloalkyl, 5-6 membered heteroaryl, and 4-7 membered heterocycloalkyl group of R^{a4}, R^{b4}, R^{c4}, and R^{d4} are each optionally substituted with 1, 2, or 3 substituents independently selected from R¹⁹;
alternatively, R^{c4} and R^{d4} together with the nitrogen atom to which they are attached form a 4-, 5-, 6-, or 7-membered heterocycloalkyl group which is optionally substituted with 1, 2 or 3 substituents independently selected from R¹⁹;
R^{e4} is H or C₁₋₄ alkyl;
R¹¹ is selected from C₁₋₆ alkyl, C₂₋₆ alkenyl, C₂₋₆ alkynyl, and C₁₋₆ haloalkyl; wherein said C₁₋₆ alkyl, C₂₋₆ alkenyl, and C₂₋₆ alkynyl are each optionally substituted with 1, 2 or 3 substituents independently selected from R¹⁹;
alternatively, R¹⁰ and R¹¹ together with the carbon atom to which they are attached form a 3-, 4-, 5-, 6-, or 7-membered cycloalkyl group or a 4-, 5-, 6-, 7-, 8-, 9-, or 10-membered heterocycloalkyl group; wherein said 3-, 4-, 5-, 6-, or 7-membered cycloalkyl group and 4-, 5-, 6-, 7-, 8-, 9-, or 10-membered heterocycloalkyl group are each optionally substituted with 1, 2, 3 or 4 R^{10A};
R¹² is H or C₁₋₄ alkyl which is optionally substituted by R¹⁷;
R¹⁷, at each occurrence, is independently selected from halo, CN, NO₂, OR^{a7}, SR^{a7}, C(O)R^{b7}, C(O)NR^{c7}R^{d7}, C(O)OR^{a7}, OC(O)R^{b7}, OC(O)NR^{c7}R^{d7}, C(═NR^{e7})NR^{c7}R^{d7}, NR^{c7}C(═NR^{e7})NR^{c7}R^{d7}, NR^{c7}R^{d7}, NR^{c7}C(O)R^{b7}, NR^{c7}C(O)OR^{a7}, NR^{c7}C(O)NR^{c7}R^{d7}, NR^{c7}S(O)R^{b7}, NR^{c7}S(O)₂R^{b7}, NR^{c7}S(O)₂NR^{c7}R^{d7}, S(O)R^{b7}, S(O)NR^{c7}R^{d7}, S(O)₂R^{b7}, S(O)₂NR^{c7}R^{d7}, C₁₋₆ alkyl, C₂₋₆ alkenyl, C₂₋₆ alkynyl, C₁₋₆ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₆ alkyl, C₂₋₆ alkenyl, C₂₋₆ alkynyl, phenyl, C₃₋₆ cycloalkyl, 5-6 membered heteroaryl, and 4-7 membered heterocycloalkyl groups of R¹⁷ are each optionally substituted with 1, 2, or 3 substituents independently selected from R¹⁹;
R^{a7}, R^{b7}, R^{c7}, and R^{d7}, at each occurrence, are independently selected from H, C₁₋₄ alkyl, C₂₋₄ alkenyl, C₂₋₄ alkynyl, C₁₋₄ haloalkyl, phenyl, C₃₋₆ cycloalkyl, a 5-6 membered heteroaryl moiety comprising carbon and 1, 2, or 3 heteroatoms independently selected from N, O and S, and a 4-7 membered heterocycloalkyl moiety comprising carbon and 1, 2, or 3 heteroatoms independently selected from N, O and S; wherein said C₁₋₄ alkyl, C₂₋₄ alkenyl, C₂₋₄ alkynyl, phenyl, C₃₋₆ cycloalkyl, 5-6 membered heteroaryl, and 4-7 membered heterocycloalkyl groups of R^{a7}, R^{b7}, R^{c7}, and R^{d7} are each optionally substituted with 1, 2, or 3 substituents independently selected from R¹⁹;
alternatively, R^{c7} and R^{d7} together with the nitrogen atom to which they are attached form a 4-, 5-, 6-, or 7-membered heterocycloalkyl group which is optionally substituted with 1, 2 or 3 substituents independently selected from R¹⁹;
R^{e7} is H or C₁₋₄ alkyl;
R¹⁹, at each occurrence, is independently selected from halo, CN, NO₂, OR^{a9}, SR^{a9}, C(O)R^{b9}, C(O)NR^{c9}R^{d9}, C(O)OR^{a9}, OC(O)R^{b9}, OC(O)NR^{c9}R^{d9}, NR^{c9}R^{d9}, NR^{c9}C(O)R^{b9}, NR^{c9}C(O)OR^{a9}, NR^{c9}C(O)NR^{c9}R^{d9}, NR^{c9}S(O)R^{b9}, NR^{c9}S(O)₂R^{b9}, NR^{c9}S(O)₂NR^{c9}R^{d9}, S(O)R^{b9}, S(O)NR^{c9}R^{d9}, S(O)₂R^{b9}, S(O)₂NR^{c9}R^{d9}, C₁₋₄ alkyl, C₂₋₄ alkenyl, C₂₋₄ alkynyl, and C₁₋₄ haloalkyl;
R^{a9}, R^{c9}, and R^{d9}, at each occurrence, are independently selected from H and C₁₋₄ alkyl; and
R^{b9} is C₁₋₄ alkyl.

3. The compound of claim 1, having Formula (II): 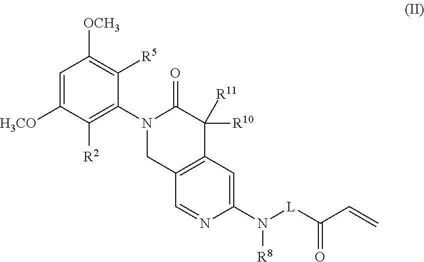
or a pharmaceutically acceptable salt thereof, wherein:
R² is F or Cl;
R⁵ is F or Cl;
L is a bond or *—CR^{7A}R^{7B} [symbol] CR^{7C}R^{7D}—NR^{7E}—, wherein the symbol * indicates the point of attachment to NR⁸ in Formula (II); wherein the symbol [symbol] represents a single or double bond; and wherein R^{7B} and R^{7D} are absent when [symbol] is a double bond; and
R⁸ is H or methyl.

4. The compound of claim 1, having Formula (V): 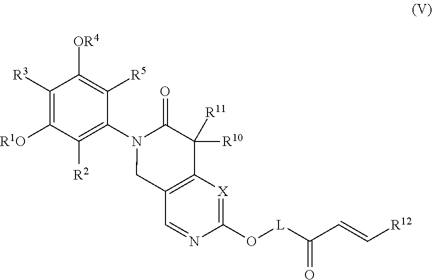
or a pharmaceutically acceptable salt thereof, wherein:
L is a bond or *—CR^{7A}R^{7B} [symbol] CR^{7C}R^{7D}—NR^{7E}—, wherein the symbol * indicates the point of attachment to —O— in Formula (V); wherein the symbol [symbol] represents a single or double bond; and wherein R^{7B} and R^{7D} are absent when [symbol] is a double bond.

5. The compound of claim 1, or a pharmaceutically acceptable salt thereof, wherein R² is F and R⁵ is F.

6. The compound of claim 1, or a pharmaceutically acceptable salt thereof, wherein R¹⁰ is C₁₋₆ alkyl and R¹¹ is C₁₋₆ alkyl.

7. The compound of claim 1, or a pharmaceutically acceptable salt thereof, wherein R¹⁰ and R¹¹ are each methyl.

8. The compound of claim 1, or a pharmaceutically acceptable salt thereof, wherein R¹⁰ and R¹¹ together with the carbon atom to which they are attached form a 3-, 4-, 5-, 6-, or 7-membered cycloalkyl group.

9. The compound of claim 1, or a pharmaceutically acceptable salt thereof, wherein R¹⁰ and R¹¹ together with the carbon atom to which they are attached form a cyclopropyl group.

10. The compound of claim 1, or a pharmaceutically acceptable salt thereof, wherein R¹⁰ and R¹¹ together with the carbon atom to which they are attached form 4-, 5-, 6-, or 7-membered heterocycloalkyl group.

11. The compound of claim 1, or a pharmaceutically acceptable salt thereof, wherein R¹⁰ and R¹¹ together with the carbon atom to which they are attached form a tetrahydropyranyl group.

12. The compound of claim 1, or a pharmaceutically acceptable salt thereof, wherein L is *—CR^{7A}R^{7B} [symbol] CR^{7C}R^{7D}—NR^{E}—, wherein the symbol * indicates the point of attachment to Y, NR⁸ or O in Formula (I′), (I), (II) or (V); wherein the symbol [symbol] represents a single or double bond; and wherein R^{7B} and R^{7D} are absent when [symbol] is a double bond.

13. The compound of claim 1, or a pharmaceutically acceptable salt thereof, wherein:
L is *—CR^{7A}R^{7B} [symbol] CR^{7C}R^{7D}—NR^{7E}—, wherein the symbol * indicates the point of attachment to Y, NR⁸ or O in Formula (I′), (I), (II) or (V); wherein the symbol [symbol] represents a single or double bond; and wherein R^{7B} and R^{7D} are absent when [symbol] is a double bond;
R^{7A} is selected from H, C₁₋₄ alkyl, C₂₋₄ alkenyl, and C₂₋₄ alkynyl;
R^{7C} is selected from H, C₁₋₄ alkyl, C₂₋₄ alkenyl, and C₂₋₄ alkynyl;
alternatively, when [symbol] is a double bond, R^{7A} and R^{7C} together with the carbon atoms to which they are attached form a phenyl group or a 5-6 membered heteroaryl moiety comprising carbon and 1, 2, or 3 heteroatoms independently selected from N, O and S, wherein said phenyl and 5-6 membered heteroaryl groups are each optionally substituted with 1, 2, or 3 substituents independently selected from R¹⁷;
alternatively, when [symbol] is a single bond, R^{7A} and R^{7C} together with the carbon atoms to which they are attached form a C₃₋₇ cycloalkyl group or a 4-7 membered heterocycloalkyl moiety comprising carbon and 1, 2, or 3 heteroatoms independently selected from N, O and S; wherein said C₃₋₇ cycloalkyl group and 4-7 membered heterocycloalkyl group are each optionally substituted with 1, 2, or 3 substituents independently selected from R¹⁷;
R^{7E} is selected from H or C₁₋₄ alkyl;
R¹⁷, at each occurrence, is independently selected from OH, CN, amino, halo, C₁₋₆ alkyl, C₁₋₄ alkoxy, C₁₋₄ alkylthio, C₁₋₄ alkylamino, di(C₁₋₄ alkyl)amino, C₁₋₄ haloalkyl, and C₁₋₄ haloalkoxy; and
R⁸ is H.

14. The compound of claim 1, or a pharmaceutically acceptable salt thereof, wherein:
L is *—CR^{7A}R^{7B} [symbol] CR^{7C}R^{7D}—NR^{7E}—, wherein the symbol * indicates the point of attachment to Y, NR⁸ or O in Formula (I′), (I), (II) or (V), and wherein the symbol [symbol] represents a single bond;
R^{7A} and R^{7C} together with the carbon atoms to which they are attached form a C₃₋₇ cycloalkyl optionally substituted with 1, 2, or 3 substituents independently from R¹⁷;
R^{7E} is selected from H or methyl;
R¹⁷, at each occurrence, is independently selected from OH, CN, amino, halo, C₁₋₆ alkyl, C₁₋₄ alkoxy, C₁₋₄ alkylthio, C₁₋₄ alkylamino, di(C₁₋₄ alkyl)amino, C₁₋₄ haloalkyl, and C₁₋₄ haloalkoxy; and
R⁸ is H.

15. The compound of claim 14, or a pharmaceutically acceptable salt thereof, wherein:
R^{7A} and R^{7C} together with the carbon atoms to which they are attached form a cyclohexyl group.

16. The compound of claim 1, or a pharmaceutically acceptable salt thereof, wherein:
L is *—CR^{7A}R^{7B} [symbol] CR⁷CR^{7D}—NR^{7E}—, wherein the symbol * indicates the point of attachment to NR⁸ in Formula (I), wherein the symbol [symbol] represents a double bond, and wherein R^{7B} and R^{7D} are absent;
R^{7A} and R^{7C} together with the carbon atoms to which they are attached form a phenyl optionally substituted with 1, 2, or 3 substituents independently from R¹⁷;
R^{7E} is selected from H or methyl;
R¹⁷, at each occurrence, is independently selected from OH, CN, amino, halo, C₁₋₆ alkyl, C₁₋₄ alkoxy, C₁₋₄ alkylthio, C₁₋₄ alkylamino, di(C₁₋₄ alkyl)amino, C₁₋₄ haloalkyl, and C₁₋₄ haloalkoxy; and
R⁸ is H.

17. The compound of claim 1, or a pharmaceutically acceptable salt thereof, wherein:
L is *—CR^{7A}R^{7B} [symbol] CR^{7C}R^{7D}—NR⁷E-, wherein the symbol * indicates the point of attachment to NR⁸ in Formula (I), and wherein the symbol [symbol] represents a single bond;
R^{7A} and R^{7C} together with the carbon atoms to which they are attached form a 4-7 membered heterocycloalkyl moiety comprising carbon and 1, 2, or 3 heteroatoms independently from N, O and S, optionally substituted with 1, 2, or 3 substituents independently selected from R¹⁷;
R^{7E} is selected from H or methyl;
R¹⁷, at each occurrence, is independently selected from OH, CN, amino, halo, C₁₆ alkyl, C₁₋₄ alkoxy, C₁₋₄ alkylthio, C₁₋₄ alkylamino, di(C₁₋₄ alkyl)amino, C₁₋₄ haloalkyl, and C₁₋₄ haloalkoxy; and
R⁸ is H.

18. The compound of claim 17, or a pharmaceutically acceptable salt thereof, wherein:
R^{7A} and R^{7C} together with the carbon atoms to which they are attached form a tetrahydrofuranyl moiety.

19. The compound of claim 1, or a pharmaceutically acceptable salt thereof, wherein L is 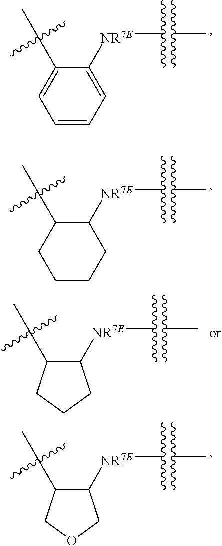
each of which is optionally substituted with from 1 to 3 R¹⁷ groups, wherein the single wavy lines indicate the point of the attachment to Y, O, or NR⁸ in formula (I′), (I), (II) or (V) and the double wavy lines indicate the point of attachment to the carbonyl group of the —C(O)CH═CHR¹² moiety in formula (I′), (I), (II) or (V).

20. The compound of claim 1, or a pharmaceutically acceptable salt thereof, wherein L is a bond.

21. The compound of claim 1 selected from:
N-(2-(2′-(2,6-difluoro-3,5-dimethoxyphenyl)-3′-oxo-2′,3′-dihydro-1′H-spiro[cyclopropane-1,4′-[2,7]naphthyridine]-6′-ylamino)phenyl)acrylamide;
N-(2′-(2,6-difluoro-3,5-dimethoxyphenyl)-3′-oxo-2′,3′-dihydro-1′H-spiro[cyclopropane-1,4′-[2,7]naphthyridine]-6′-yl)-N-methylacrylamide;
N-(2-(2′-(2,6-difluoro-3,5-dimethoxyphenyl)-3′-oxo-2′,3′-dihydro-1′H-spiro[cyclopropane-1,4′-[2,7]naphthyridine]-6′-ylamino)cyclohexyl)acrylamide;
N-(4-(2′-(2,6-difluoro-3,5-dimethoxyphenyl)-3′-oxo-2′,3′-dihydro-1′H-spiro[cyclopropane-1,4′-[2,7]naphthyridine]-6′-ylamino)tetrahydrofuran-3-yl)acrylamide;
N-(2-(2′-(2,6-difluoro-3,5-dimethoxyphenyl)-3′-oxo-2′,3′-dihydro-1′H-spiro[cyclopropane-1,4′-[2,7]naphthyridine]-6′-ylamino)-3-methylphenyl)acrylamide;
N-(2-(7-(2,6-difluoro-3,5-dimethoxyphenyl)-5,5-dimethyl-6-oxo-5,6,7,8-tetrahydro-2,7-naphthyridin-3-ylamino)phenyl)acrylamide;
N-(2-(2′-(2,6-difluoro-3,5-dimethoxyphenyl)-3′-oxo-2′,3′-dihydro-1′H-spiro[cyclopentane-1,4′-[2,7]naphthyridine]-6′-ylamino)phenyl)acrylamide; and
N-(2-(2-(2,6-difluoro-3,5-dimethoxyphenyl)-3-oxo-2,2′,3,3′,5′,6′-hexahydro-1H-spiro[[2,7]naphthyridine-4,4′-pyran]-6-ylamino)phenyl)acrylamide;
or a pharmaceutically acceptable salt of any of the aforementioned.

22. The compound of claim 1 selected from:
N-(2-(2′-(2,6-difluoro-3,5-dimethoxyphenyl)-3′-oxo-2′,3′,4,5-tetrahydro-1′H,2H-spiro[furan-3,4′-[2,7]naphthyridine]-6′-ylamino)phenyl)acrylamide;
N-(2-{[2′-(2,6-difluoro-3,5-dimethoxyphenyl)-3′-oxo-2′,3′-dihydro-1′H-spiro[cyclopropane-1,4′-[2,7]naphthyridin]-6′-yl]amino}-4-morpholin-4-ylphenyl)acrylamide;
N-(2-(2′-(2,6-difluoro-3,5-dimethoxyphenyl)-3′-oxo-2′,3′-dihydro-1′H-spiro[cyclopropane-1,4′-[2,7]naphthyridine]-6′-ylamino)-4-(4-methylpiperazin-1-yl)phenyl)acrylamide; and
N—((1 S,2R)-2-(6′-(2,6-difluoro-3,5-dimethoxyphenyl)-7′-oxo-6′,7′-dihydro-5′H-spiro[cyclopropane-1,8′-pyrido[4,3-d]pyrimidine]-2′-yloxy)cyclopentyl)acrylamide;
or a pharmaceutically acceptable salt of any of the aforementioned.

23. A pharmaceutical composition comprising a compound of claim 1, or a pharmaceutically acceptable salt thereof, and at least one pharmaceutically acceptable carrier.

24. A method of inhibiting an FGFR4 enzyme comprising contacting said enzyme with a compound of claim 1, or a pharmaceutically acceptable salt thereof.

25. A method of treating cancer in a patient comprising administering to said patient a therapeutically effective amount of a compound of claim 1, or a pharmaceutically acceptable salt thereof.

26. The method of claim 25 wherein said cancer is selected from hepatocellular cancer, bladder cancer, breast cancer, cervical cancer, colorectal cancer, endometrial cancer, gastric cancer, head and neck cancer, kidney cancer, liver cancer, lung cancer, ovarian cancer, prostate cancer, esophageal cancer, gall bladder cancer, pancreatic cancer, thyroid cancer, skin cancer, leukemia, multiple myeloma, chronic lymphocytic lymphoma, adult T cell leukemia, B-cell lymphoma, acute myelogenous leukemia, Hodgkin's or non-Hodgkin's lymphoma, Waldenstrom's Macroglubulinemia, hairy cell lymphoma, Burkett's lymphoma, glioblastoma, melanoma, and rhabdosarcoma.

27. The method of claim 25 wherein said cancer is selected from hepatocellular cancer, breast cancer, bladder cancer, colorectal cancer, melanoma, mesothelioma, lung cancer, prostate cancer, pancreatic cancer, testicular cancer, thyroid cancer, squamous cell carcinoma, glioblastoma, neuroblastoma, uterine cancer, and rhabdosarcoma.


SEQUENCE LISTING (1 sequence)

SEQ ID NO: 1 - protein, 15 aa, Artificial Sequence
  synthetic peptide
EQEDEPEGDYFEWLE